(19) United States
(12) Patent Application Publication
(10) Pub. No.: US 2015/0072416 A1
(43) Pub. Date: Mar. 12, 2015

(54) METABOLITE FOR IMPROVING PRODUCTION, MAINTENANCE AND PROLIFERATION OF PLURIPOTENT STEM CELLS, COMPOSITION COMPRISING THE SAME, AND METHOD OF CULTURING PLURIPOTENT STEM CELL USING THE SAME

(71) Applicant: KOREA RESEARCH INSTITUTE OF BIOSCIENCE AND BIOTENOLOGY, Daejeon (KR)

(72) Inventors: Yee Sook Cho, Daejeon (KR); Myung Jin Son, Daejeon (KR); Mi Young Son, Daejeon (KR)

(73) Assignee: Korea Research Institute of Bioscience and Biotech, Daejeon (KR)

(21) Appl. No.: 14/358,652

(22) PCT Filed: Apr. 26, 2013
(86) PCT No.: PCT/KR2013/003655

§ 371 Date: May 15, 2014

(30) Foreign Application Priority Data
May 29, 2012 (KR) 10-2012-0056810

(51) Int. Cl. C12N 5/074

(52) U.S. Cl. CPC C12N 5/0607; C12N 2501/999
USPC 435/366; 546/316; 435/377

(57) ABSTRACT

According to the present invention, when nicotinamide is added in a culture process for producing pluripotent stem cells from human differentiated cells, it can increase the efficiency of reprogramming and can significantly reduce the time required for induction of reprogramming. It was verified that nicotinamide inhibits the induction of senescence and oxidative stress in the reprogramming process and increases cell proliferation and mitochondrial activity to effectively improve culture conditions for induction of reprogramming. Particularly, the present invention will contribute to optimizing a process of producing induced pluripotent stem cells from a small amount of patient-specific somatic cells obtained from various sources, and thus it will significantly improve a process of developing clinically applicable personalized stem cell therapy agents and new drugs and will facilitate the practical application of these agents and drugs. In another aspect, according to the present invention, in defined culture conditions in which feeder cells and serum were not used, it was found that nicotinamide can provide a culture medium composition effective for maintaining the undifferentiated state of human embryonic stem cells and human induced pluripotent stem cells, which are typical pluripotent stem cells. The invention can be effectively used for the development of a high-efficiency system for culturing large amounts of human pluripotent stem cells, which is required for the industrialization of human pluripotent stem cells.

### TECHNICAL FIELD

The present invention relates to a composition comprising nicotinamide effective for promoting the reprogramming of differentiated cells/somatic cells into pluripotent stem cells, a cell culture comprising the same, a method of producing reprogrammed pluripotent stem cells using the same, and a method for maintaining and culturing pluripotent stem cells including the reprogrammed pluripotent stem cells in an undifferentiated state. Moreover, the present invention relates to a composition comprising nicotinamide for improving the mitochondrial function of pluripotent stem cells, which is involved in the cell fate determination of the pluripotent stem cells, and a cell culture comprising the composition.

### BACKGROUND ART

Stem cells generally refers to cells that have excellent self-renewal potential while maintaining an undifferentiated state and are capable of differentiating in a tissue-specific manner so as to have certain functions and shapes under certain environments and conditions. Human pluripotent stem cells, including human embryonic stem cells and human induced pluripotent stem cells, are capable of self-renewal under suitable in vitro culture conditions and have a pluripotent ability to differentiate into all types of cells of the body. Pluripotent stem cells include embryonic stem cells and induced pluripotent stem cells. /Due to such characteristics, the results of studies on these pluripotent stem cells have been applied not only for the understanding of biological basic knowledge, including the development, differentiation and growth of organisms, but also for the development of cell therapy agents for fundamental treatment of various diseases and the development of new drugs. While efforts have been increasingly made to develop practically applicable technology based on human pluripotent stem cells in various fields, there are still problems to be solved in terms of efficiency, safety and economy in a process for the production and proliferative culture of human pluripotent stem cells. Specifically, it is required to develop a technology for producing large amounts of undifferentiated and differentiated stem cells, which can satisfy the demand for the stem cells at any time. Particularly, for the development of cell therapy agents, it is necessary to ensure cell culture technology, which has excellent performance, can provide clinically applicable cells and is highly efficient.

Reprogramming technology that produces induced pluripotent stem cells (iPSCs) (reprogrammed stem cells) having self-renewal and pluripotent properties by dedifferentiation/reprogramming of differentiated somatic cells in an in vitro culture process was first proven successful in mouse cells and human cells in the years 2006 and 2007, respectively, by professor Yamanaka's team (Kyoto University, Japan) (Cell, 126: 663-676, 2006; Cell, 131:1-12, 2007). Since then, there has been intense competition between countries in the world to advance the practical application of stem cell-based therapeutic agents and new drugs on the basis of the reprogramming technology (Takahashi et al, Cell, 2007; Yu et al, Science, 2007; Park et al, Cell, 2008).

The success of development of the reprogramming technology by professor Yamanaka's team provided a springboard for remarkable development of strategies for establishing autologous pluripotent stem cell lines from patient's somatic cells, and the reprogramming technology is recognized as the best solution for addressing bioethical issues and immune compatibility that can be caused by the use of human embryonic stem cells, thereby providing infinite possibilities for its future application to regenerative medical fields. Particularly, the reprogramming technology makes it possible to produce stem cells having the same properties as those of human embryonic stem cells from autologous somatic cells that are obtainable in a relatively easy way without causing particular damage to a patient. Thus, the reprogramming technology is recognized as a technology capable of supplying cell resources that are most useful for the development of patient-specific stem cell therapeutic agents.

Because the reprogramming technology is currently being rapidly developed, it is expected that the demand for and application of iPSCs or tissue-specific differentiated cells derived from iPSCs in the fields of new drug development and fusion technology will infinitely increase.

However, current reprogramming technology that overexpresses the embryonic stem cell-specific transcription factors Oct4, Sox2, c-Myc and Klf4 genes as reprogramming factors to reprogram differentiated human somatic cells into multipotent/pluripotent induced stem cells shows a very low reprogramming efficiency of about 0.01-0.1%. Thus, in order to satisfy the demand for cell therapeutic agents, there is an urgent need for the development of a variety of reprogramming factors capable of significantly improving the reprogramming efficiency of somatic cells together with the development of a technology enabling the use of these reprogramming factors in a reprogramming process.

Generally, undifferentiated human pluripotent stem cells can be continuously cultured by co-culturing with feeder cells such as mouse embryonic fibroblasts (MEFs) or in feeder-free conditions using conditioned media (CM) obtained from cultures of MEFs or chemically defined medium. However, co-culture with animal feeder cells or the use of conditioned media from animal feeder cells involves the risk of transmitting one or more infectious agents such as viruses to human pluripotent stem cells. Because one of the purposes of culture of human pluripotent stem cells is to produce tissue that can be eventually transplanted into the human body, it is required that stem cells have never been exposed to other kinds of cells or media used in culture of other kinds of cells, due to the above-described risk.

Despite a rapid increase in the demand for human pluripotent stem cells, difficulty in the technology and method for maintaining and culturing stem cells in an undifferentiated state acts as an obstacle in the development of related technologies. Particularly, in order to use human pluripotent stem cells as cell therapeutic agents, the development of media containing no animal-derived factors and the development of mass culture systems that satisfy the demand for human pluripotent stem cells are very important.

In recent years, there have been continued efforts to develop a method of culturing human pluripotent stem cells without animal feeder cells and sera or a method of culturing human pluripotent stem cells only using defined factors, and this method has been recognized to have high economic added value.

### DISCLOSURE

### Technical Problem

Under such circumstances, the present inventors have made extensive efforts to discover a new pluripotency factor that is effective for maintaining and culturing human pluripotent stem cells in an undifferentiated state and inducing the reprogramming of human somatic cells into human pluripotent stem cells, and as a result, have verified that, when nicotinamide playing an important role in the cellular metabolic process is added to a culture medium at a suitable concentration, it significantly will increase the efficiency of reprogramming into human pluripotent stem cells and is effective for maintaining and culturing human pluripotent stem cells in an undifferentiated state, thereby completing the present invention.

### Technical Solution

It is an object of the present invention to provide a composition comprising nicotinamide for promoting reprogramming of differentiated cells into pluripotent stem cells.

Another object of the present invention is to provide a method of producing reprogrammed pluripotent stem cells from differentiated cells.

Still another object of the present invention is to provide a method of culturing reprogrammed pluripotent stem cells in an undifferentiated state.

Still another object of the present invention is to provide a composition comprising nicotinamide for improving the mitochondrial function of pluripotent stem cells.

Still another object of the present invention is to provide a composition comprising nicotinamide for maintaining pluripotent stem cells in an undifferentiated state.

Still another object of the present invention is to provide a method of culturing pluripotent stem cells so as to be maintained in an undifferentiated state.

### Advantageous Effects

According to the present invention, when nicotinamide is added in a culture process for producing reprogrammed pluripotent stem cells from human differentiated cells, it can increase the efficiency of reprogramming and can significantly reduce the time required for the induction of reprogramming. It was verified that nicotinamide inhibits the induction of senescence and oxidative stress in the reprogramming process and increases cell proliferation and mitochondrial activity to effectively improve culture conditions for induction of reprogramming. Particularly, the present invention will contribute to optimizing a process of producing induced pluripotent stem cells from a small amount of patient-specific somatic cells obtained from various sources, and thus it will significantly improve a process of developing clinically applicable personalized stem cell therapy agents and new drugs and will facilitate the practical application of these agents and drugs. In another aspect, according to the present invention, in defined culture conditions in which feeder cells and serum were not used, it was found that nicotinamide can provide a culture medium composition effective for maintaining the undifferentiated state of human embryonic stem cells and human induced pluripotent stem cells, which are typical pluripotent stem cells. The present invention can be effectively used for the development of a high-efficiency system for culturing large amounts of human pluripotent stem cells, which is required for the industrialization of human pluripotent stem cells.

### DESCRIPTION OF DRAWINGS

FIG. 1 shows the results of analysis of the expression of major enzymes in the NAD⁺ (nicotinamide adenine dinucleotide) biosynthesis process in embryonic stem cells and induced pluripotent stem cells. FIG. 1a shows microarray assay results, in which enzymes whose expression in undifferentiated stem cells increased are indicated by the arrow “↑”, and enzymes whose expression decreased is indicated by the arrow “↓”. FIG. 1b indicates the increase or decrease in expression of NAD⁺ biosynthesis-related enzymes in undifferentiated (Un) and differentiated (Diff) embryonic stem cells and induced pluripotent stem cells by the difference in number of arrows “↑” or “↓”. FIG. 1c shows the results of analyzing the expression of major enzymes by real-time polymerase chain reaction (values are expressed as mean±S.E.; p<0.05, ** p<0.01, determined by t-test).

FIG. 2 shows the results of examining the effects of nicotinamide and an NAD precursor in embryonic stem cells treated with the NAD⁺ synthesis inhibitor FK866 during culture. FIG. 2Aa shows the results of measuring the concentration of NAD in embryonic stem cells treated with FK866 and an NAD precursor, and FIG. 2Ab shows the results of quantifying AP activity under this condition. FIG. 2Ba shows the results of quantifying the number of cells, and FIG. 2Bb shows the results of quantifying the apoptosis level (values are expressed as mean±S.E.; * p<0.05, ** p<0.01, determined by t-test).

FIG. 3 shows the results of examining the effect of nicotinamide in cells treated with the NAD⁺ synthesis inhibitor FK866 under a culture condition of mTeSR1 chemically defined medium (mTeSR1 CDM). FIG. 3A shows the results of measuring the concentration of NAD in embryonic stem cells treated with FK866 and nicotinamide under the mTeSR1 culture condition, and FIG. 3B shows the results of quantifying AP activity under the condition (values are expressed as mean±S.E.; p<0.05, determined by t-test).

FIG. 4 shows the results of examining the efficiency of reprogramming of human fibroblasts into induced pluripotent stem cells under culture conditions without feeder cells. FIG. 4A shows the results of examining the increase in reprogramming efficiency by various NAD precursors. FIG. 4B shows the results of examining the increase in reprogramming efficiency by nicotinamide under various concentration conditions. The upper panels are AP staining images of induced pluripotent stem cell colonies, and the lower panels indicate the number of ES-like colonies and the number of AP-positive colonies (values are expressed as mean±S.E.; * p<0.05, ** p<0.01, determined by t-test).

FIG. 5 shows the increase in efficiency of reprogramming into induced pluripotent stem cells as a function of the time of treatment with nicotinamide. FIG. 5a shows the results obtained by adding 1 mM of nicotinamide during the indicated periods of time in a reprogramming process, seeding the cells on Matrigel, and then performing AP staining on day 21. The number of AP-positive colonies is indicated and the values are expressed as mean±S.E. FIG. 5b shows the results obtained by introducing reprogramming factors, culturing the cells for 4 days while adding 1 mM of nicotinamide thereto, and then measuring the number of viable cells. FIG. 5c shows the results obtained by introducing reprogramming factors, seeding Matrigel with either cells to which nicotinamide was added for 5 days or the same number of cells to which no nicotinamide was added, adding nicotinamide to the cells for 21 days, and measuring reprogramming efficiency by AP staining of the cells to which nicotinamide was added or not added. The number of AP-positive colonies is indicated and the values are expressed as mean±S.E. (* p<0.05, ** p<0.01, determined by t-test).

FIG. 6 shows the results of the immunostaining reaction of the stem cell markers Nanog and Tra-1-60 according to treatment with nicotinamide at different time points of reprogramming (left). The right figure of FIG. 6 shows the results of quantifying immunostained clusters, and the values are expressed as mean±S.E. (Control; nicotinamide-treated group—Nam; scale bar=200 μm; *p<0.05, ** p<0.01, determined by t-test).

FIG. 7 shows the results of quantifying the mRNA expression levels of the stem cell marker Nanog and the proliferation regulatory factor TERT by real-time polymerase chain reaction at different time points of reprogramming, and the values are expressed as mean±S.E. (Control; nicotinamide-treated group—Nam; *p<0.05, determined by t-test).

FIG. 8 shows the results of quantifying the time required to obtain hiPSCs colonies, and the values are expressed as mean±S.E. (Control; nicotinamide-treated group—Nam; ** p<0.01, determined by t-test).

FIG. 9 shows the results of examining the epigenetic regulatory effect of nicotinamide. The results were obtained by performing chromatin immunoprecipitation with antibodies to methylated lysine residues 4 and 27 of histone 3, amplifying the promoter regions of the pluripotency factors Nanog and Oct4, and quantifying the increase or decrease in the expression of the factors. Under culture conditions with nicotinamide and NAD precursor somatic cells (hFFs) and induced pluripotent stem cells (hiPSCs) were used as controls (values are expressed as mean±S.E.; * p<0.05, ** p<0.01, determined by t-test).

FIG. 10 shows the results of examining the cell growth efficiency caused by addition of nicotinamide during reprogramming of human fibroblasts into induced pluripotent stem cells. The number of viable cells was measured after adding 0-10 mM of nicotinamide to human fibroblasts and human fibroblasts transfected with reprogramming factors (Oct4(O), Sox2(S), c-Myc(M), and Klf4(K)). The upper panel shows cell images, and the lower panel shows values expressed as mean±S.E. (scale bar=500 μm; *p<0.05, ** p<0.01, determined by t-test).

FIG. 11 shows the cell proliferation efficiency caused by addition of nicotinamide during reprogramming of human fibroblasts into induced pluripotent stem cells. The proliferation rate of cells was measured using BrdU incorporation after adding 0-10 mM of nicotinamide to human fibroblasts and human fibroblasts transfected with reprogramming factors (Oct4(O), Sox2(S), c-Myc(M), and Klf4(K)). The upper panel shows representative images of BrdU⁺ cells (scale bar=500 μm). The lower panel shows the results of quantifying the relative number of BrdU⁺ cells per well and calculating the ratio (%) of the measured cell number relative to the total number of cells. The data are expressed as mean±SE (n=3) (* p<0.05, ** p<0.01, determined by t-test).

FIG. 12 shows the results of examining the ratio of proliferating cells using a live cell imaging method. The upper panel shows the results of quantifying the ratio of resting-stage cells (red) and dividing cells (yellow) on day 12 of induction of reprogramming, and the value are expressed as mean±S.E. (Control; nicotinamide-treated group—Nam; scale bar=100 μm, *** p<0.001, determined by t-test).

FIG. 13 shows the results of senescence-associated β-galactosidase (SA-β-gal) staining. FIG. 13A shows the results obtained by staining cells with senescence-associated β-galactosidase on day 26 of induction of reprogramming, and then immunostaining the cells with the stem cell markers Nanog and Tra-1-60 (upper) and quantifying the ratio of cells stained with senescence-associated β-galactosidase (lower). FIG. 13B shows senescence-associated heterochromatin foci (SAHF) and indicates the results obtained by staining cells with senescence-associated β-galactosidase on day 26 of induction of reprogramming, and then staining the DNA with DAPI (4′,6-diamidino-2-phenylindole) (upper) and quantifying the ratio of cells stained with β-galactosidase (green) and having damaged DNA (blue) (lower), and the values are expressed as mean±S.E. (Control; nicotinamide-treated group—Nam; ** p<0.01, *** p<0.001, determined by t-test).

FIG. 14 shows the results of measurement of reactive oxygen species (ROS). The degrees of formation of reactive oxygen species in a nicotinamide-treated group and an untreated control group on day 19 of induction of reprogramming were measured by flow cytometry (upper) and quantified (lower), and the values are expressed as mean±S.E. Hydrogen peroxide (H₂O₂) was used as a control for the formation of reactive oxygen species (** p<0.01, determined by t-test).

FIG. 15 shows a comparison of protein damage caused by oxidative stress between a control group and a nicotinamide-treated group (Nam) at different time points of reprogramming (upper). The values obtained by quantifying the degree of damage to somatic cells (hFFs) relative to 1 after normalization to the expression level of beta-actin are expressed as mean S.E. (lower) (*p<0.05, ** p<0.01, determined by t-test).

FIG. 16 shows the results of measuring mitochondrial membrane potential by fluorostaining the mitochondrial membrane potential in a control group and a nicotinamide-treated group (Nam) at different time points of reprogramming (upper), measuring the mitochondrial membrane potential by flow cytometry (left lower) and quantifying the relative ratio (right lower), and the values are expressed as mean±S.E. (** p<0.01, *** p<0.001, determined t-test).

FIG. 17 shows the results of analyzing the expression of cellular senescence/apoptosis signaling factors by immunostaining. Nanog, Tra-1-60, pp53, p53, p27 and p21 in a nicotinamide-treated group and an untreated group were immunostained on day 19 of induction of reprogramming, and the nuclei were stained with DAPI (blue).

FIG. 18 shows the results of analyzing the expression of cellular senescence/apoptosis signaling factors by Western blot analysis. The protein expression levels of each factor in a nicotinamide-treated group and an untreated group at different time points of reprogramming were analyzed (left), and the bands were quantified (right) (values are expressed as ±S.E.; * p<0.05, ** p<0.01, determined by t-test).

FIG. 19 shows the change in mRNA expression level of cell signaling factors caused by nicotinamide. The mRNA expression levels of p53 and p21 in a nicotinamide-treated group and an untreated group on day 19 of induction of reprogramming were analyzed by real-time polymerase chain reaction and compared with the expression level of β-actin, and the values are expressed as mean±S.E. (*p<0.05, determined by t-test).

FIG. 20 shows the results of analyzing the expression of p53 and p21 at varying concentrations of nicotinamide. The difference of protein expression level of each factor between nicotinamide concentrations was analyzed by Western blotting at different time points of reprogramming.

FIG. 21 shows the results of analyzing the expression of human stem cell markers in an induced pluripotent stem cell line (Nam-iPS), induced from human fibroblasts by addition of nicotinamide, using an immunostaining method (scale bar=200 μm).

FIG. 22 shows the results of analyzing the expression of stem cell marker genes and reprogramming factors in an induced pluripotent stem cell line (Nam-iPS), induced from human fibroblasts by addition of nicotinamide, using RT-PCR. Semi-quantitative RT-PCR was performed using transgene-specific PCR primers enabling the determination of relative expression levels between total, endogenous (Endo) and retrovirus expression (Trans) genes.

FIG. 23 shows the results of analyzing the insertion of genes into the genome of an induced pluripotent stem cell line (Nam-iPS) induced from human fibroblasts by addition of nicotinamide.

FIG. 24 shows the results of analyzing the promoter methylation patterns of the transcription factors Oct4 and Nanog in an induced pluripotent stem cell line (Nam-iPS) induced from human fibroblasts by addition of nicotinamide, H9 human embryonic stem cells (hESs) and human fibroblasts (hFFs). Each horizontal line of circles indicates an individual sequence from one amplicon. The empty circle and the black circle indicate demethylated and methylated CpG respectively, and the ratio (%) of methylated CpG is shown.

FIG. 25 shows the gene expression profiles of an induced pluripotent stem cells (Nam-iPS) reprogrammed from human fibroblasts by addition of nicotinamide. FIG. 25A shows the results obtained by analyzing the heat map and hierarchical clustering of general gene expression in Nam-iPS, H9 human embryonic stem cells (hESs) and human fibroblasts (hFFs), calculating the Pearson correlation, and performing hierarchical clustering by the average linkage clustering method. The distance was calculated by GeneSpring GX7.3.1 for comparisons between different cell lines and is indicated above the tree lines. The color bar indicates the color code gene expression in log 2 scale. FIG. 25B shows scatter plots indicating a comparison of gene expression profile between Nam-iPS, hES and hFF. The stem cell marker genes OCT4, SOX2, c-Myc, KLF4, NANOG, LIN28 and REX1 are indicated.

FIG. 26 shows RT-PCR images indicating that an induced pluripotent stem cells (Nam-iPS) reprogrammed from human fibroblasts by addition of nicotinamide conserves the ability to differentiate into three germ layers by formation of embryoid bodies.

FIG. 27 shows immunocytochemical images indicating that an induced pluripotent stem cells (Nam-iPS) reprogrammed from human fibroblasts by addition of nicotinamide conserves the ability to differentiate into three germ layers by formation of embryoid bodies.

FIG. 28 shows images indicating that an induced pluripotent stem cell line (Nam-iPS), induced from human fibroblasts by addition of nicotinamide forms teratomas that demonstrates the in vivo differentiation potential of the cell line.

FIG. 29 shows the results of culturing human embryonic stem cells in culture media containing various NAD precursors without using feeder cells. Specifically, it shows AP staining images of H9 human embryonic stem cells cultured in MEF-CM (conditioned medium) or UM (unconditioned medium) (left) and the results of densitometric analysis performed to determine the relative amounts of AP-positive colonies (right), and the values are expressed as mean±S.E. 0.1 mM of each of Nam, L-Trp, NA, Iso-Nam, 3-ABA and NAD and 10 μM of NMN were used (* p<0.05, determined by t-test).

FIG. 30 shows the results of culturing human embryonic stem cells (H9 hES) and human induced pluripotent stem cells (hiPS) in culture media containing various concentrations of nicotinamide without using feeder cells. Specifically, it shows AP staining images of stem cells cultured in MEF-CM or UM (upper) and the relative amounts of AP positive colonies determined by densitometric analysis (lower), and the values are expressed as mean±S.E. (* p<0.05, ** p<0.01, determined by t-test).

FIG. 31 shows the results of culturing human embryonic stem cells (H9 and H1) and human induced pluripotent stem cells (hiPS) in culture media containing various concentrations of nicotinamide in the presence or absence of feeder cells. In the presence of feeder cells, UM was used, and in the absence of feeder cells, mTeSR1 CDM was used. Specifically, FIG. 31 shows AP staining images (left) and the relative amounts of AP positive colonies determined by densitometric analysis (right), and the values are expressed as mean±S.E. (* p<0.05, ** p<0.01, determined by t-test).

FIG. 32 shows the results of examining the effect of nicotinamide on the promotion of proliferation of human embryonic stem cells. In a culture condition in which no feeder cells were used, the indicated concentration of nicotinamide was added to mTeSR1 CDM, and the proliferation rate of H9 human embryonic stem cells was measured using BrdU incorporation. The upper panel shows representative images of BrdU⁺ cells (bar=500 μm). The lower panel shows the results of quantifying the relative number of BrdU⁺ cells per field and calculating the ratio of the measured cell number to the total cell number. The data are expressed as mean±S.E. (n=3) (** p<0.01, determined by t-test).

FIG. 33 shows the results of examining the effect of nicotinamide on the improvement in the mitochondrial function of human embryonic stem cells. In a culture condition in which no feeder cells were used, the indicated concentration of nicotinamide was added to mTeSR1 CDM, and the mitochondrial membrane potential of H9 human embryonic stem cells was measured using the JC-1 staining method. Activated mitochondria are stained with red, and non-activated mitochondria are stained with green. The upper panel shows representative images of cells stained with JC-1 (bar=20 μm). The middle panel shows the fluorescence intensity profiles of JC-1 staining. The lower panel shows the red/green ratio after JC-1 staining, expressed as a value relative to that of an untreated control group. The data are expressed as mean±S.E. (n=3) (** p<0.01, determined by t-test).

FIG. 34 shows the results of examining whether the undifferentiated state of human embryonic stem cells could be maintained when the cells were treated with nicotinamide and the NAD⁺ precursors nicotinic acid (NA) and NAD⁺ in UM medium capable of easily inducing a differentiated state (FIG. 34a). FIG. 34aA shows AP staining images of undifferentiated human embryonic stem cells, and the lower panel indicates the number of AP-positive colonies, and the values are expressed as mean±S.E. (* p<0.05, ** p<0.01, determined by t-test). FIG. 34Ab shows the results of measurement of the NAD level. FIG. 34b shows the results of low-density assay. FIG. 34bA shows AP staining images of undifferentiated human embryonic stem cells and induced pluripotent stem cells, which were treated with NA and NAD′ after single-cell dissociation, and FIG. 34bB shows the results of measurement of the apoptosis level.

FIG. 35 shows the results of TRA-1-60 staining, which indicate that cells can be subcultured for a long period of time for 10 or more passages in an undifferentiated state in UM medium containing 0.1 mM nicotinamide.

FIG. 36 shows the results of karyotyping after long-term subculture in nicotinamide-containing medium.

### BEST MODE

In one aspect, the present invention provides a composition for promoting reprogramming of differentiated cells into pluripotent stem cells, which comprises nicotinamide.

As used herein, the term “nicotinamide (Nam or vitamin B3) refers to a nicotinic acid amide that a complex of water-soluble vitamin with vitamin B. Nicotinamide having a molecular formula of C₆H₆N₂O is present as the coenzymes nicotinamide nucleotide, NAD⁺ and NADP⁺ in vivo and is involved in many oxidation/reduction reactions. Nicotinic acid amide is used as an agent for treatment of chronic alcoholism, angina pectoris, frostbite and the like and is abundantly present in livers, fishes, grain embryos, yeast, beans and meats. Nicotinamide is vitamin that is colorless crystalline powder, and it is produced from tryptophan, like nicotinic acid, and widely distributed in animals and plants. Pellagra that is nicotinamide deficiency causes dermatitis, diarrhea, delirium, anxiety and the like and leads to death, and when it is excessively taken, it causes hepatotoxicity, gastric ulcer and diabetes. As used herein, the term “NAD⁺” means nicotinamide adenine dinucleotide, and the term “NADP⁺” means nicotinamide adenine dinucleotide phosphate. Meanwhile, in an example of the present invention, nicotinic acid (NA) that is a precursor of NAD⁺ was used.

As used herein, the term “differentiation” refers to a phenomenon in which the structure or function of cells is specialized during the division, proliferation and growth thereof. That is, the term refers to a process in which the feature or function of cell or tissue of an organism changes in order to perform work given to the cell or tissue. For example, a process in which pluripotent stem cells such as embryonic stem cells change to ectoderm, mesoderm and endoderm stem cells is also defined as differentiation, and in a narrow sense, a process in which hematopoietic stem cells change to red blood cells, white blood cells, platelets or the like also corresponds to differentiation.

As used herein, the term “differentiated cells” refers to cells that undergone the differentiation process so as to have a specific shape and function. Differentiated cells that are used in the present invention are not specifically limited, but are preferably somatic cells or progenitor cells. In addition, differentiated cells are preferably cells of human origin.

As used herein, the term “somatic cells” refers to any differentiated cells other than germ cells, which constitute animals or plants and have a chromosome number of 2n.

As used herein, the term “progenitor cells” refers to undifferentiated progenitor cells which do not express a differentiated differentiation phenotype when their progeny cells express a specific differentiated phenotype. For example, progenitor cells for neurons are neuroblasts, and progenitor cells for myotube cells are myoblasts.

As used herein, the term “pluripotent stem cells” refers to cells that are capable of differentiating into all the tissues of the body and have self-renewal potential, but is not limited thereto. Pluripotent stem cells in the present invention include those derived from humans, monkeys, pigs, horses, cattle, sheep, dogs, cats, mice, rabbits or the like. Preferably, pluripotent stem cells are pluripotent stem cells of human origin.

As used herein, the term “embryonic stem cells” refers to pluripotent cells that are obtained by in vitro culture of inner cell masses extracted from blastocysts immediately before implantation into the uterus of the mother, are capable of differentiating into all the tissues of the body, and have self-renewal potential. In a broad sense, the term also includes embryoid bodies derived from embryonic stem cells. Embryonic stem cells in the present invention include embryonic stem cells derived from humans, monkeys, pigs, horses, cattle, sheep, dogs, cats, mice, rabbits or the like, and are preferably embryonic stem cells of human origin.

As used herein, the term “induced pluripotent stem cells” refers to cells reprogrammed from differentiated cells by an artificial reprogramming process so as to have pluripotent differentiation potential and is also referred to as reprogrammed stem cells. The artificial reprogramming process may be performed by the use of a virus-mediated vector such as retrovirus and lentivirus or a nonviral vector or by introduction of nonvirus-mediated reprogramming factors using proteins and cell extracts, or includes a reprogramming process that is performed by stem cell extracts, compounds or the like. Induced pluripotent stem cells have properties almost similar to those of embryonic stem cells. Specifically, induced pluripotent stem cells show similarity in cell morphology and expression patterns of gene and protein to those of embryonic stem cells, have pluripotency in vitro and in vivo, form teratomas, and generate chimeric mice upon injection into mouse blastocysts, and are capable of germline transmission. Induced pluripotent stem cells in the present invention include those derived from any animals, including humans, monkeys, pigs, horses, cows, sheep, dogs, cats, mice, rabbits, etc., and are preferably induced pluripotent stem cells of human origin.

As used herein, the term “reprogramming” or “dedifferentiation” refers to a process in which differentiated cells can be restored into cells having a new type of differentiation potential. In the present invention, the term “reprogramming” is used in the same meaning as cell reprogramming. This cell reprogramming mechanism involves the removal of epigenetic (DNA state associated with changes in gene function that occur without a change in the nucleotide sequence) marks in the nucleus, followed by establishment of a different set of marks, and different cells and tissues acquire different gene expression programs during the differentiation and growth of multicellular organisms.

As used herein, the term “promoting reprogramming” means increasing the rate of reprogramming or the efficiency of reprogramming in the reprogramming process. That is, the term includes increasing the efficiency of reprogramming in terms of speed and rate.

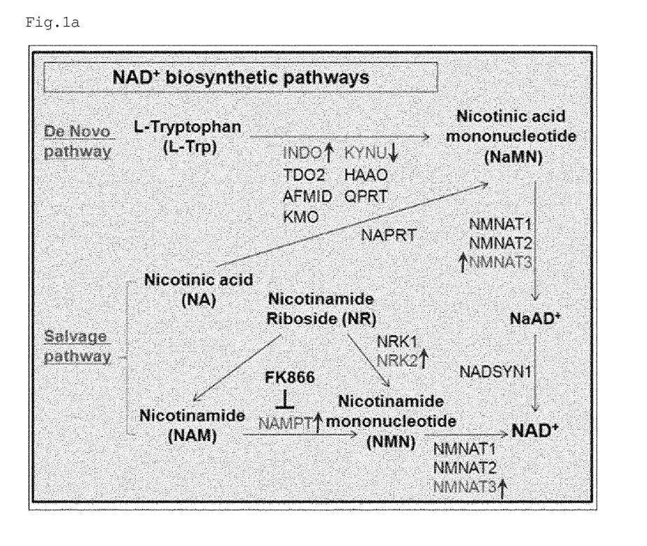

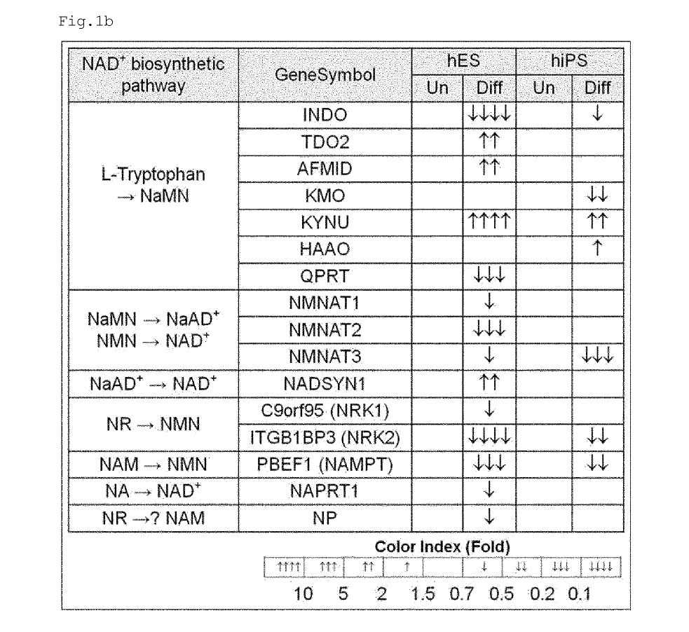

In an example of the present invention, the expression levels of major enzymes in embryonic stem cells and induced pluripotent stem cells in the NAD⁺ biosynthesis process were analyzed by microarray and real-time polymerase chain reaction (FIG. 1). As a result, it was shown that the expression levels of NAD⁺ biosynthesis-related enzymes in undifferentiated embryonic stem cells and induced pluripotent stem cells increased, and the expression levels thereof decreased when the cells differentiated (FIG. 1).

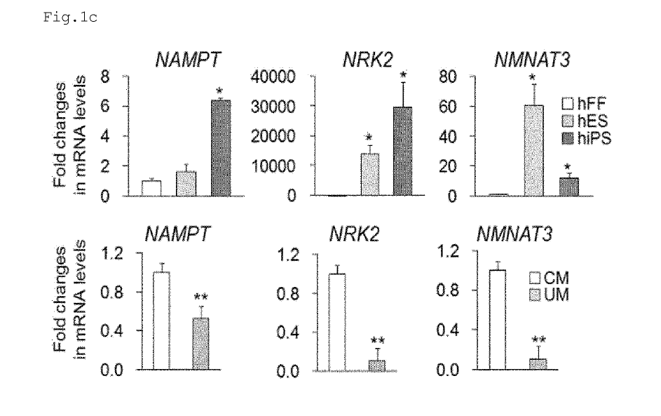

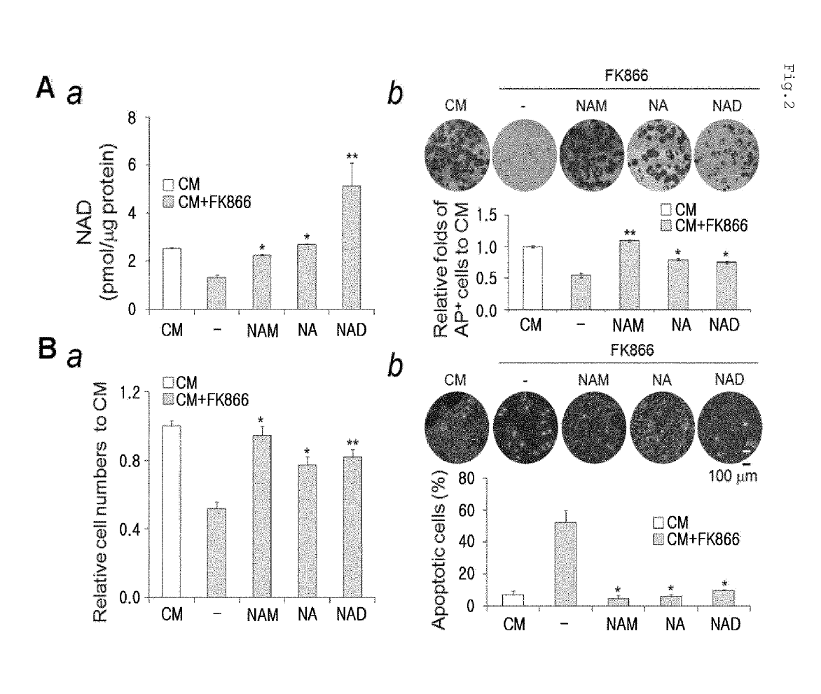

In addition, it was shown that, when cells were treated with the NAD⁺ synthesis inhibitor FK866, the concentration of NAD in the cells was decreased, apoptosis was induced and the embryonic stem cells were differentiated (FIG. 2). However, when cells were treated with FK866 together with nicotinamide or the NAD precursors nicotinic acid (NA) and NAD, the cells were recovered from damage caused by FK866 (FIG. 2), and this effect was also observed in a mTeSR1 chemically defined medium (mTeSR1-CDM) condition (FIG. 3).

A composition according to the present invention is preferably in the form of culture medium. Thus, substances that are generally contained in cell culture media may be added to the composition of the present invention, as long as they do not interfere with reprogramming of differentiated cells into pluripotent stem cells.

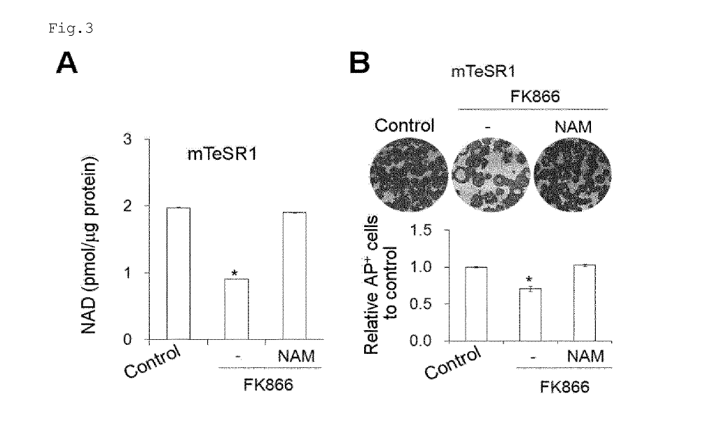

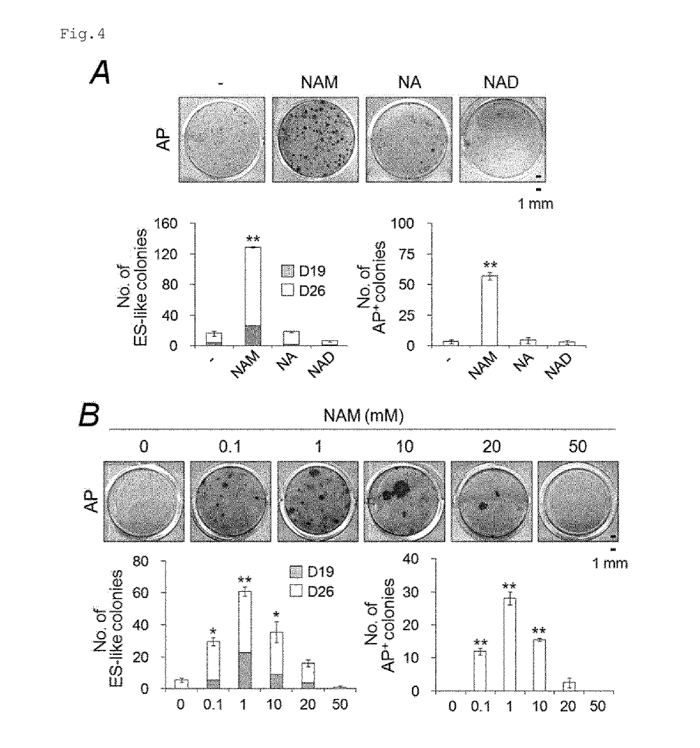

A composition for promoting reprogramming of differentiated cells into pluripotent stem cells according to the present invention comprises nicotinamide at a concentration that does not impair the survival and function of cells. Preferably, the composition of the present invention may comprise nicotinamide at a concentration of 0.01-20 mM. More preferably, it may comprise nicotinamide at a concentration of 0.05-10 mM. Most preferably, it may comprise nicotinamide at a concentration of 0.1-5 mM at a concentration (FIG. 4).

The composition for promoting reprogramming of differentiated cells into pluripotent stem cells according to the present invention comprises may comprise one or more reprogramming factors. As used herein, the term “reprogramming factor” refers to a material that induces the reprogramming of differentiated cells into induced pluripotent stem cells having a new type of differentiation potential. The reprogramming factor may be any material that induces the reprogramming of differentiated stem cells, and it may be selected depending on the kind of cells to differentiate. Preferably, the reprogramming factor that is used in the composition of the present invention may be one or more proteins selected from the group consisting of Oct4, Sox2, K1F4, c-Myc, Nanog, Lin-28 and Rex1 or one or more nucleic acid molecules encoding these proteins. More preferably, the reprogramming factor may be Oct4 protein or a nucleotide molecule encoding the protein. Particularly, the composition may comprise Oct4, Sox2, K1F4 and c-Myc proteins or nucleic acid molecules encoding these proteins.

In the present invention, the “nucleic acid molecule encoding the protein” may be operably linked to a promoter or the like so that it can express the corresponding protein by itself when being delivered into cells. In a broad sense, the term “nucleic acid molecules” may include nucleic acid molecules that can express the corresponding proteins when being inserted into the chromosome of cells. For example, nucleic acid molecules encoding one or more proteins selected from the group consisting of Oct4, Sox2, K1F4, c-Myc, Nanog, Lin-28 and Rex1 as reprogramming factors may be operably linked to an expression vector and may be delivered into cells or delivered into the chromosome of host cells.

In an example of the present invention, nucleic acid molecules encoding the reprogramming factors Oct4, Sox2, Klf4 and c-Myc were transfected into human fibroblasts by retrovirus at an MOI of 1 to induce reprogramming of the cells. In addition, nicotinamide and other NAD⁺ precursors were added at different concentrations and different time points, and the change in reprogramming efficiency caused by addition of these substances was observed. As a result, it could be seen that the addition of nicotinamide increased reprogramming efficiency by about 17 times compared to the addition of other substances (FIG. 4).

Reprogramming efficiency was determined by measuring the number of colonies showing a positive response in the pluripotency-specific marker alkaline phosphatase (AP) staining and having hESC-like morphology.

In another example of the present invention, in order to determine an optimal nicotinamide concentration range effective for increasing reprogramming efficiency, reprogramming efficiency was measured at different concentrations of nicotinamide. As a result, it was shown that reprogramming efficiency increased in a manner dependent on the concentration of nicotinamide.

Specifically, it could be seen that the reprogramming efficiency of the group treated with nicotinamide increased by 13 times (0.1 mM), 28 times (1 mM), 16 times (10 mM) and 2 times (20 mM) compared to a control group not treated with nicotinamide (FIG. 4).

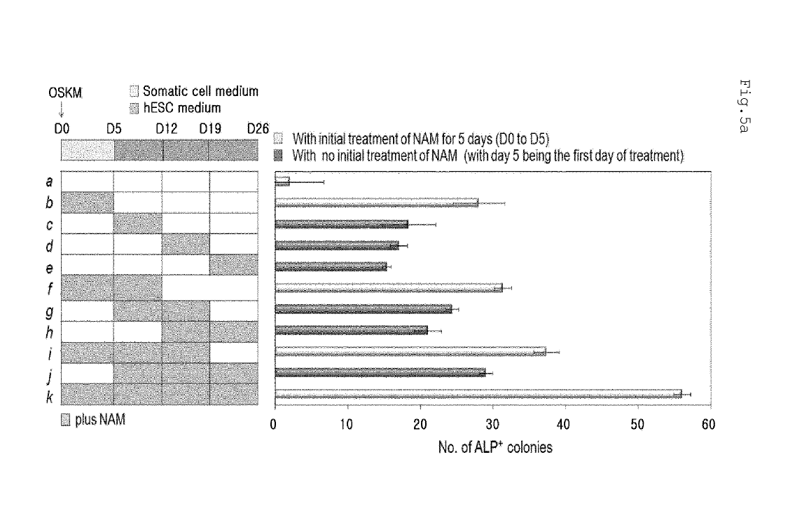

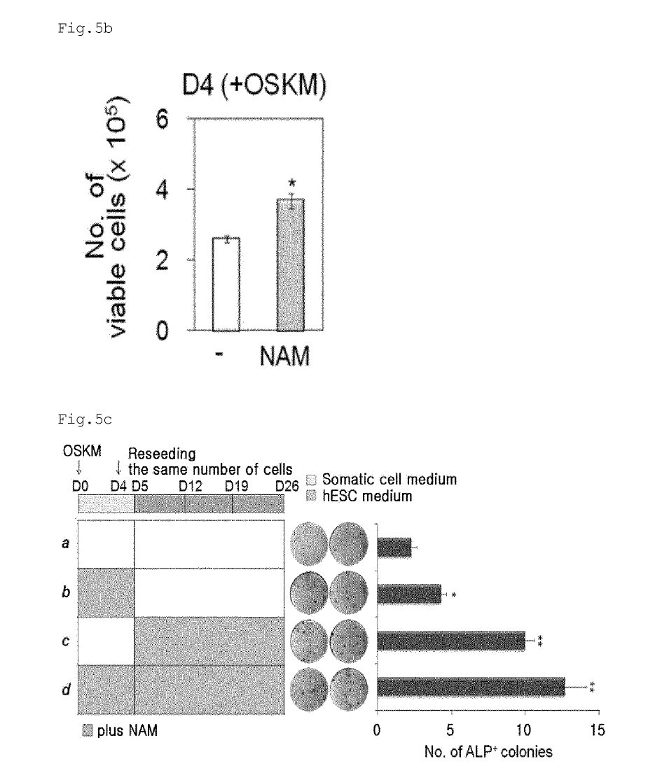

In another example of the present invention, in order to optimize the timing and period of treatment with nicotinamide, the change in reprogramming efficiency was measured after treatment with nicotinamide under various conditions. As a result, it was shown that, when treatment with nicotinamide was performed for 5-7 days in each of four divided steps consisting of step 1 (5 days after viral infection; condition b in FIG. 5A), step 2 (5-12 days after viral infection; condition c in FIG. 5A), step 3 (12-19 days after viral infection; condition d in FIG. 5A) and step 4 (19-26 days after viral infection; condition e in FIG. 5A), treatment with nicotinamide together with differentiated-cell culture medium in step 1 (5 days immediately after viral infection) most effectively increased the efficiency of reprogramming compared to 7-day treatment in the other three steps (FIG. 5A). In addition, it was shown that, when treatment with nicotinamide was performed for 12-14 days in each of three divided steps consisting of step 1 (12 days after viral infection; condition f in FIG. 5A), step 2 (5-19 days after viral infection; condition g in FIG. 5A) and step 3 (12-26 days after viral infection; condition h in FIG. 5A), treatment with nicotinamide for 12 days immediately after viral infection in step 1 (culture for 5 days in differentiated-cell culture medium, and then additional culture for 7 days in human embryonic stem cell culture medium in a fresh culture dish) most effectively increased the efficiency of reprogramming compared to 14-day treatment in the other two steps (FIG. 5A). In addition, it was shown that, when treatment with nicotinamide was performed for 19-21 days in each of two divided steps consisting of step 1 (19 days after viral infection; condition i in step 5A) and step 2 (5-26 days after viral infection; condition j in FIG. 5A), treatment with nicotinamide for 19 days after viral infection in step 1 (culture for 5 days in differentiated-cell culture medium, and then additional culture for 14 days in human embryonic stem cell culture medium in a fresh culture dish) effectively increased the efficiency of reprogramming compared to the case in which the infected cells were treated with nicotinamide while they were cultured for 21 days in human embryonic stem cell culture medium in a fresh culture dish (FIG. 5A). Among all the conditions tested, continuous treatment with nicotinamide throughout the process of inducing reprogramming showed the highest increase in the efficiency of reprogramming (condition k in FIG. 5A and condition d in FIG. 5C). In conclusion, it was verified that, when treatment with nicotinamide is performed in the initial stage of the reprogramming process, it shows the best effect on the induction of reprogramming, and even when treatment with nicotinamide is performed after the initial stage of the reprogramming process, it can significantly increase the efficiency of reprogramming in a manner dependent on the time of treatment.

In order to further analyze the effect of initial treatment with nicotinamide on an increase in the efficiency of reprogramming, a reprogramming factor was introduced into cells, and then nicotinamide was added to one group of the cells for 5 days and was not added to another group of the cells. Then, each of the two cell groups having the same cell number was re-seeded on Matrigel, and nicotinamide was added to one subgroup of each cell group for 21 days and was not added to another subgroup. Then, the possibility of the change in reprogramming efficiency with a change in cell proliferation was observed. It could be seen that the cell group treated with nicotinamide in the initial stage after infection showed an increase in reprogramming efficiency compared to the untreated control group, even when nicotinamide was not added to the cell group after reseeding (FIG. 5C: b), suggesting that the promotion of cell growth in the initial stage is effective for increasing the efficiency of reprogramming. In addition, it could be seen that the cell group treated with nicotinamide after reseeding showed an increase in reprogramming efficiency compared to the untreated group, even when nicotinamide was not added thereto in the initial stage (FIG. 5C: c), suggesting that nicotinamide is effective for promoting cell growth not only in the initial stage, but also after reseeding. This indicates that nicotinamide is associated with reprogramming efficiency.

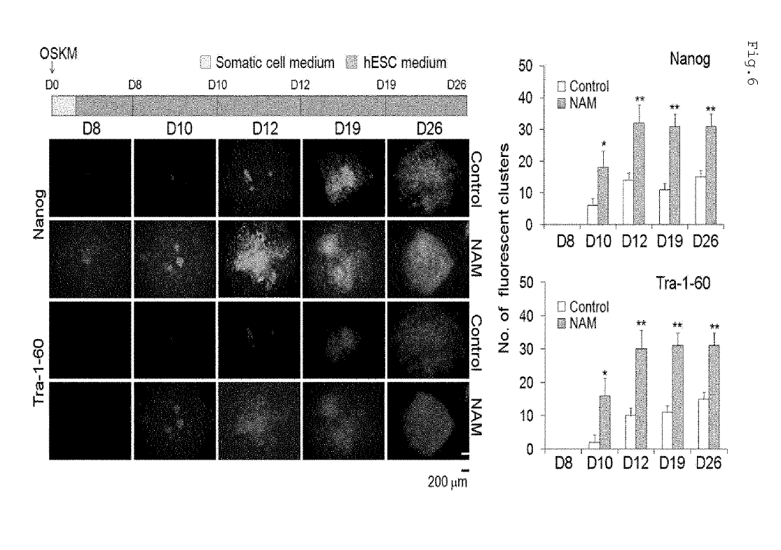

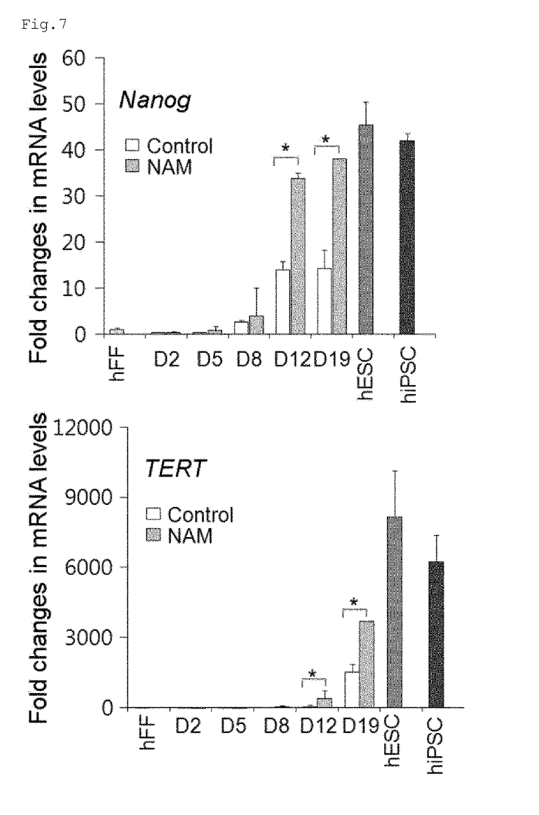

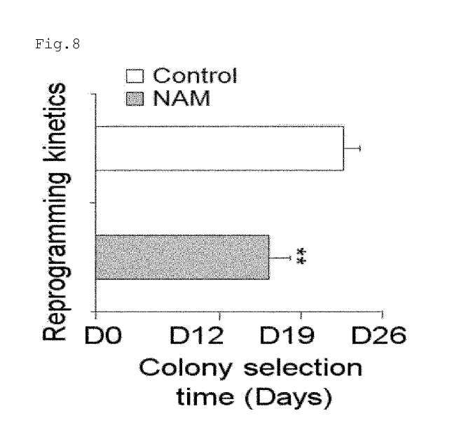

In an example of the present invention, in order to examine whether nicotinamide can promote kinetics in a culture process for reprogramming to shorten the time required for the induction of reprogramming, the expression patterns of pluripotency-specific markers were analyzed at various time points. As can be seen in FIGS. 6 and 7, the results of immunostaining analysis (FIG. 6) and real-time PCR analysis (FIG. 7) indicated that, when reprogramming was induced in a culture medium treated with nicotinamide, the expression of pluripotency-specific markers (Nanog, Tra1-81, and TERT) appeared earlier than that in the untreated control group. Also, the results of AP staining indicated that AP-positive colonies having hESC-like morphology appeared earlier in the cell group treated with nicotinamide than in the untreated control group (FIG. 8). These results suggest that nicotinamide can stimulate reprogramming kinetics to effectively shorten the time required for the induction of reprogramming.

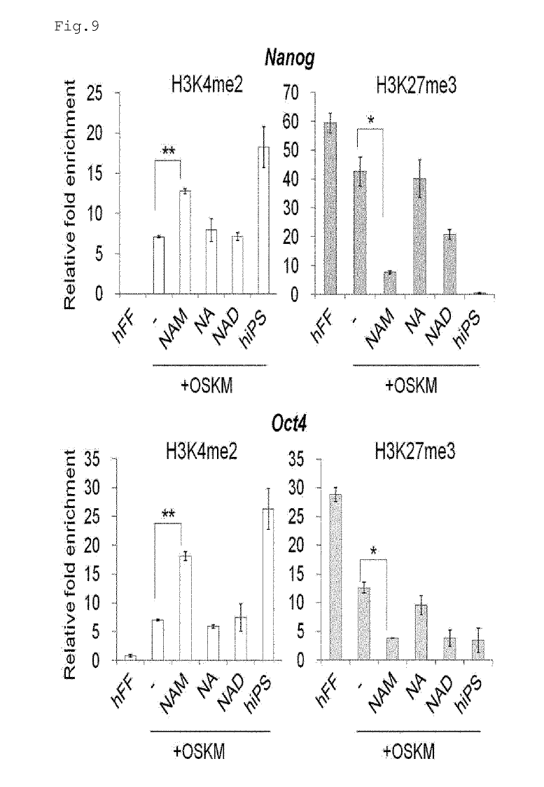

In this process, the epigenetic regulatory effect of nicotinamide was also analyzed using chromatin immunoprecipitation (FIG. 9). It was shown that methylation of lysine residue 4 of histone 3 in the promoter regions of the pluripotency factors Nanog and Oct4, which is involved in the promotion of gene expression, was increased by nicotinamide, and methylation of residue 27 which is involved in the inhibition of gene expression was decreased (FIG. 9).

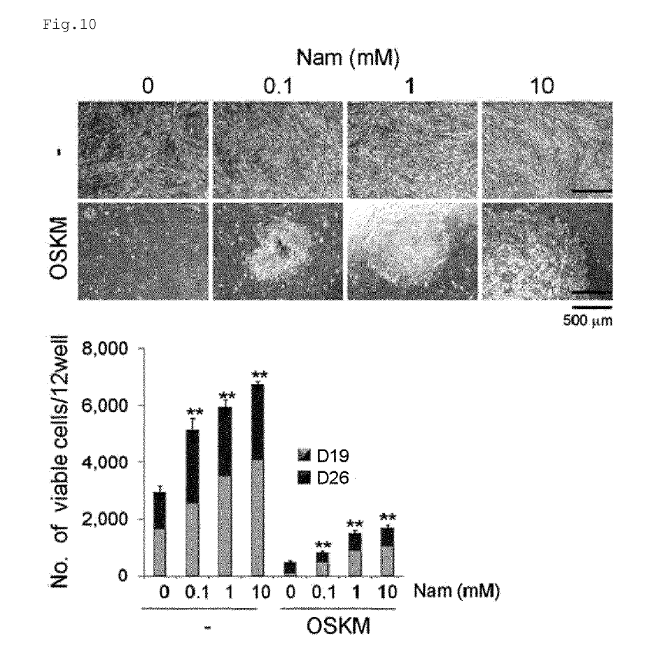

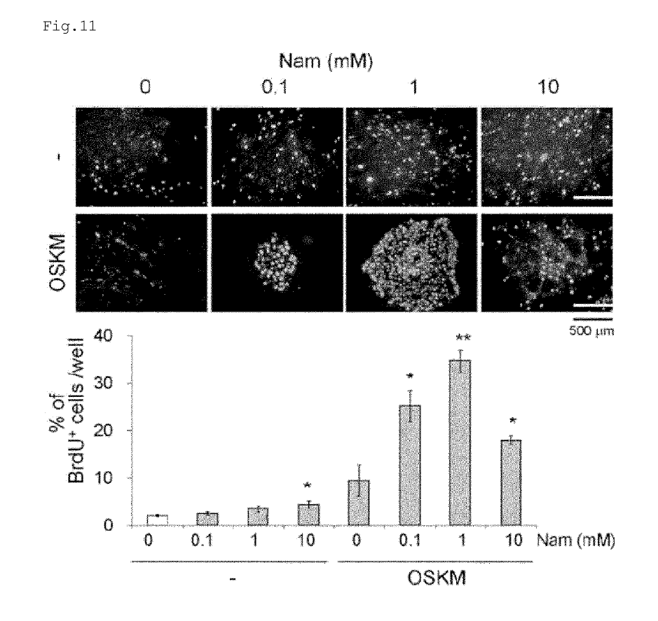

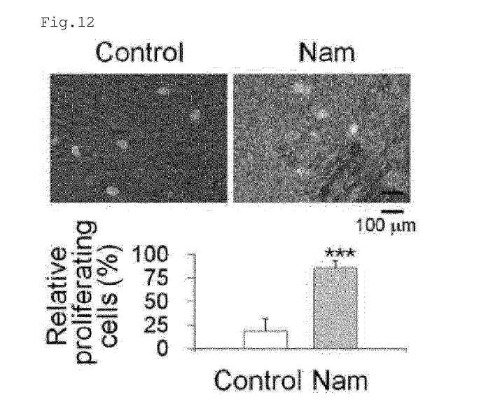

In another example of the present invention, reprogramming was induced under the conditions treated with nicotinamide at different concentrations and time points, and the total cell number was counted. As a result, it was found that nicotinamide had the effect of promoting the growth and proliferation of cells in the culture process for inducing reprogramming (FIG. 10). It was shown that an increase in the number of cells transduced with OSKM was significantly slow compared to that of a control group not transduced with OSKM and that treatment with nicotinamide under the same conditions promoted the growth of the cells (FIG. 10). In addition, a change in the proliferation of cells during a culture process for inducing reprogramming was examined by a BrdU assay (FIG. 11), and the cell cycle was examined by live-cell imaging (FIG. 12). As a result, it was observed that treatment with nicotinamide significantly improved the growth of the cells in a manner dependent on the concentration of nicotinamide, similar to the above-described results. At this time, it was shown that an optimal nicotinamide concentration for promoting cell growth was 1 mM (FIGS. 10 and 11).

In another example of the present invention, in order to examine whether nicotinamide can alleviate senescence that is induced by OSKM transduction known as an obstacle in the reprogramming process, the senescence markers senescence-associated β-galactosidase (SA-β-gal) and senescence-associated heterochromatin foci (SAHF) were analyzed. As a result, it was shown that the activity of SA-β-gal and the formation of SAHF decreased in a culture medium treated with nicotinamide (FIG. 13).

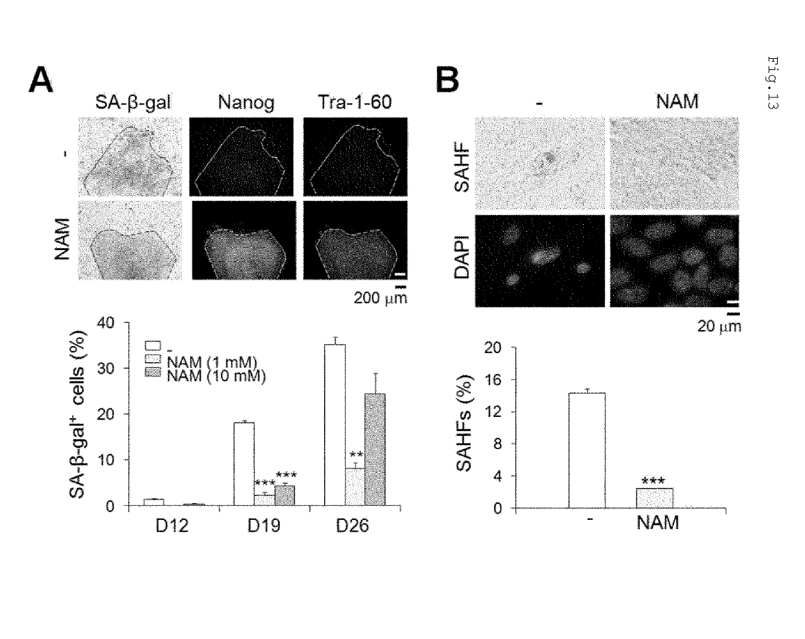

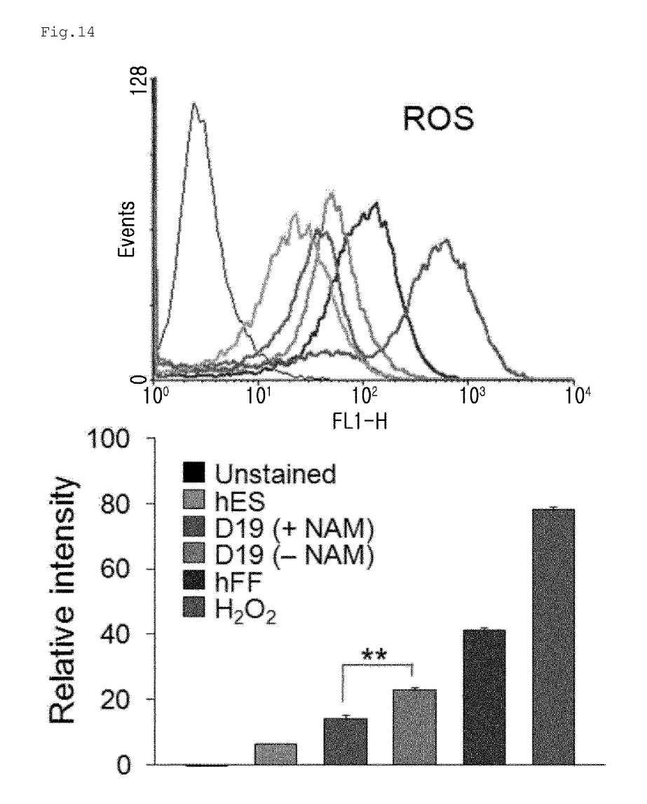

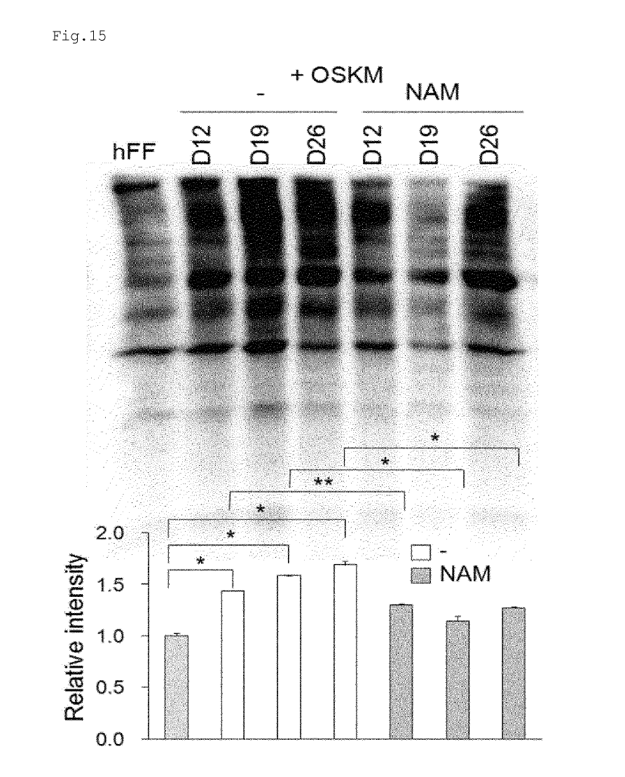

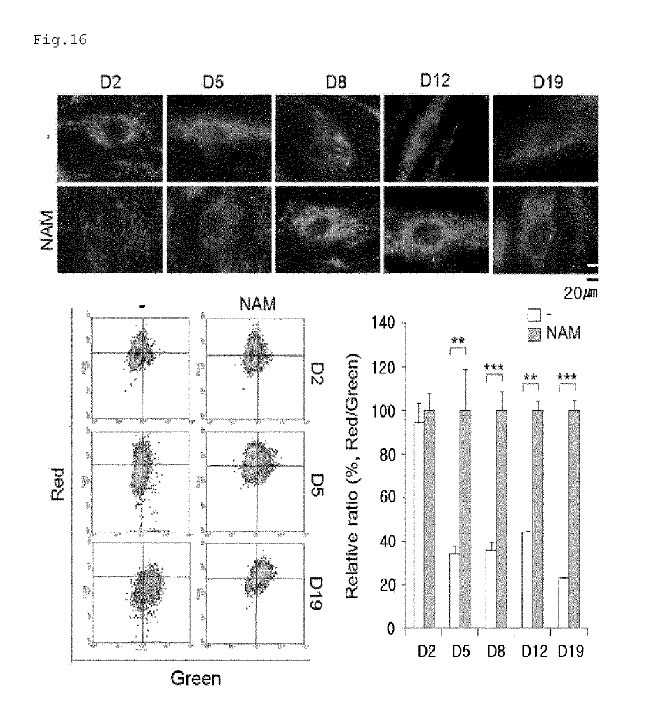

In still another example of the present invention, analysis was performed to examine whether nicotinamide influences changes in oxidative stress, known as another obstacle in the reprogramming process, and in mitochondrial activity. As a result, it was shown that nicotinamide reduced the intracellular ROS level that was increased by OSKM transduction (FIG. 14), and nicotinamide reduced the protein oxidation level (FIG. 15) and increased the reduced mitochondrial activity (FIG. 16).

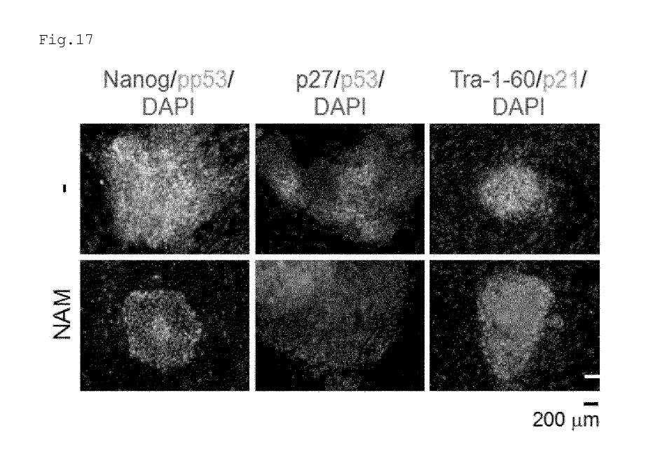

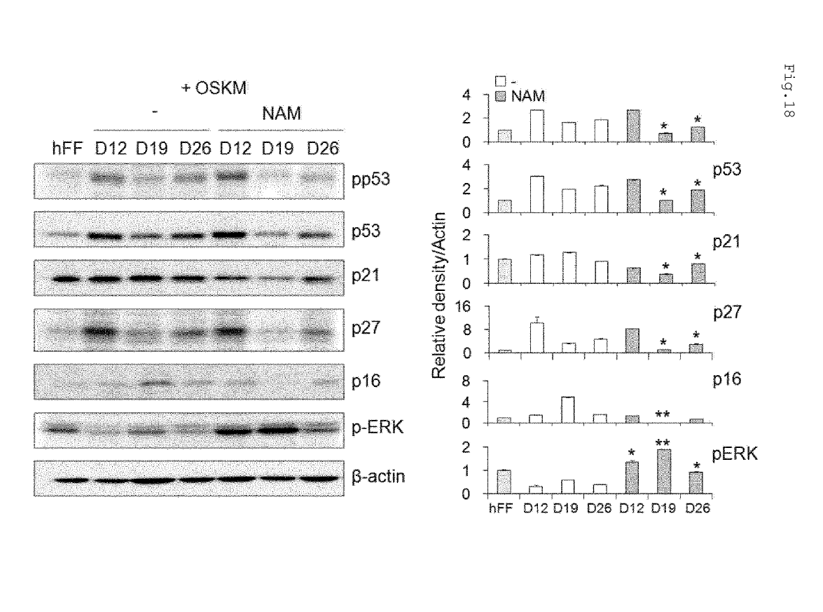

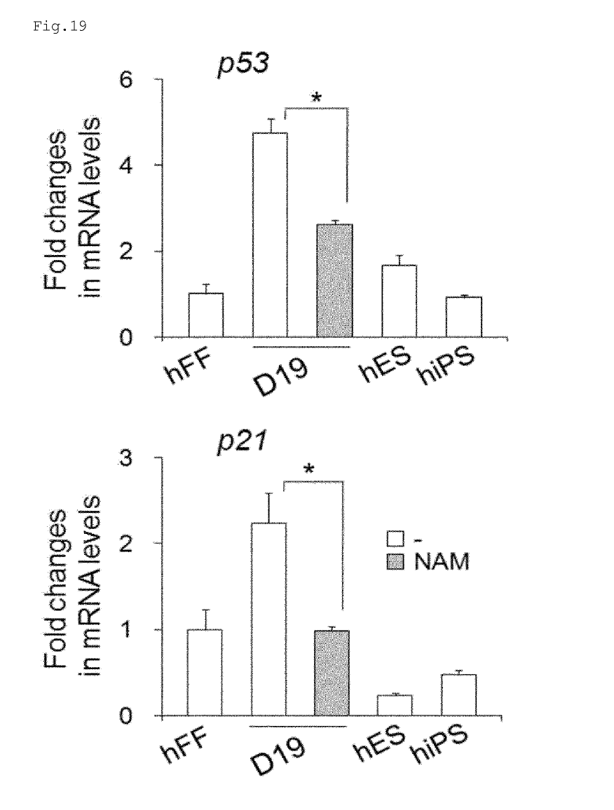

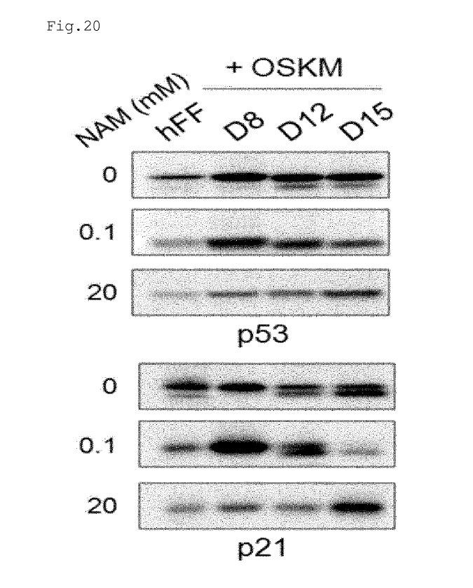

In still another example of the present invention, changes in the expression patterns of the known senescence/apoptosis signaling factors p53, p21 and p16 were observed. As can be seen in FIG. 17 (immunostaining), FIG. 18 (Western blotting) and FIG. 19 (real-time polymerase chain reaction), the expression of the senescence factors p53, p27, p21 and p16 was effectively inhibited in a culture medium treated with nicotinamide, and this effect did not appear at a high concentration (20 mM) of nicotinamide (FIG. 20). In conclusion, it was verified that nicotinamide contributes to increasing the efficiency of reprogramming by effectively inhibiting the cell senescence and cell apoptosis events that are induced in the reprogramming process.

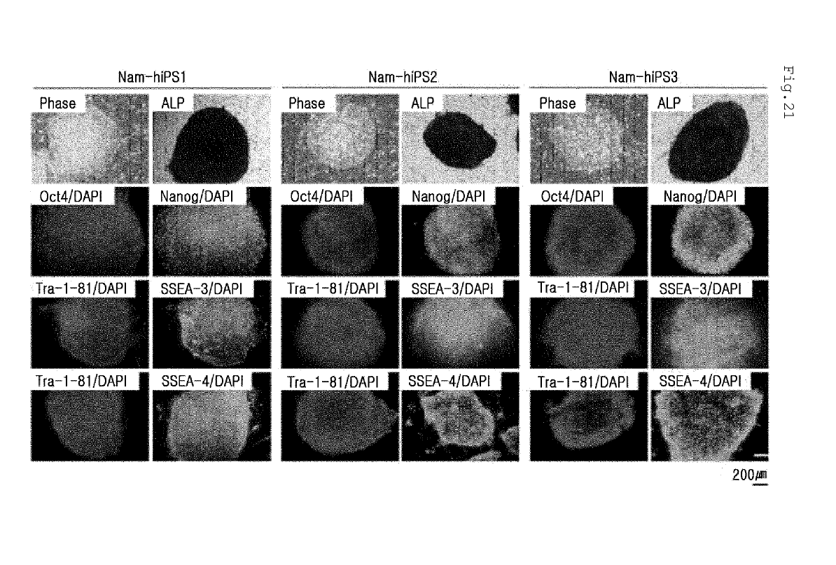

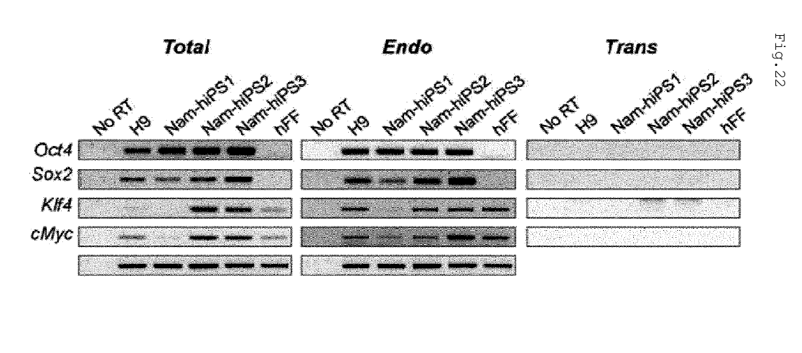

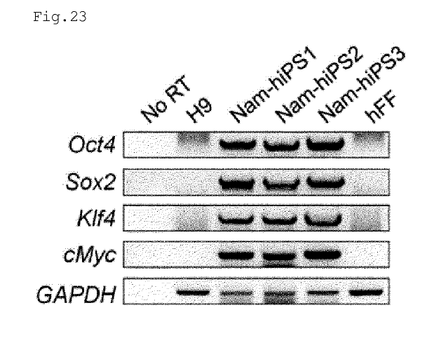

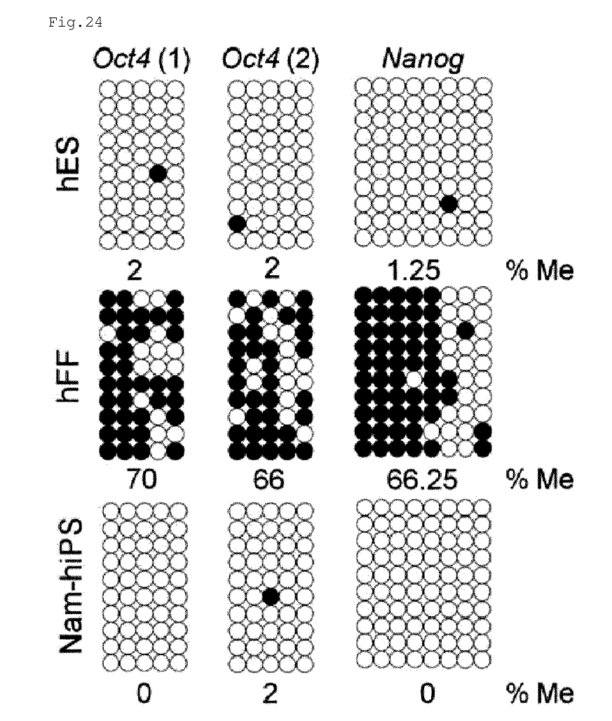

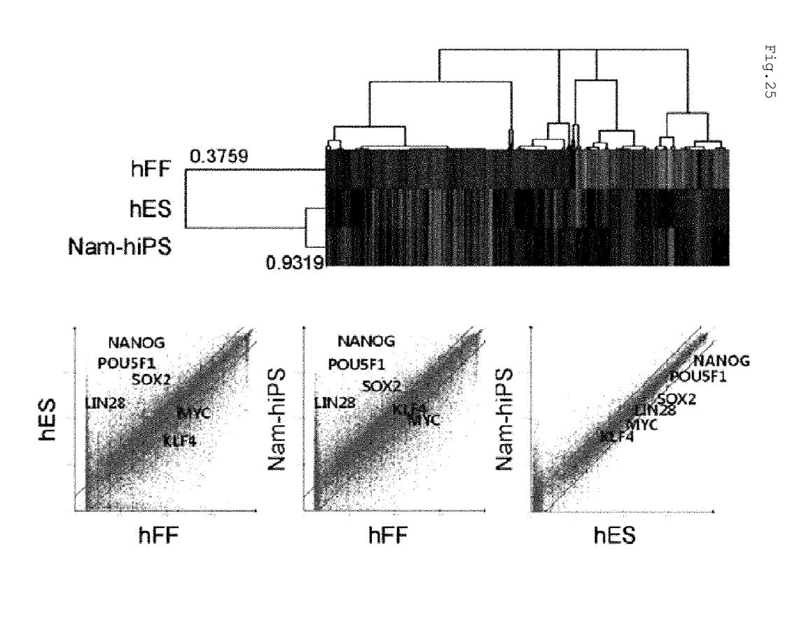

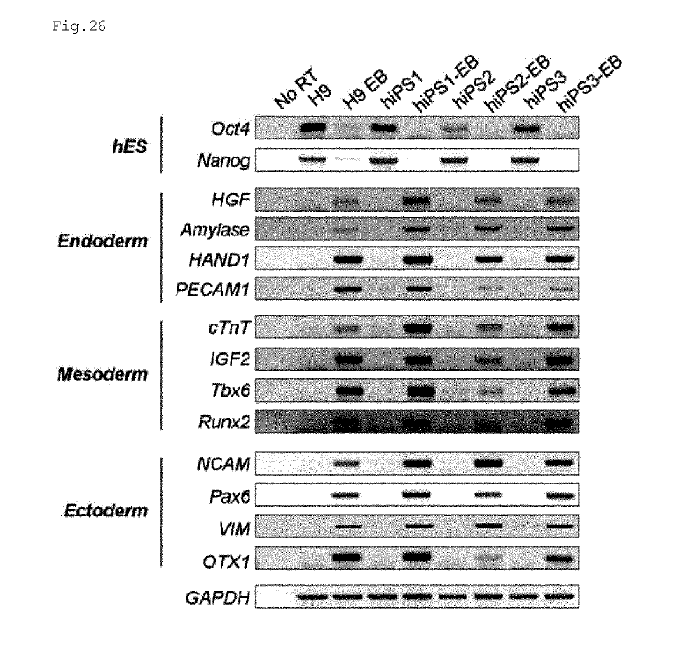

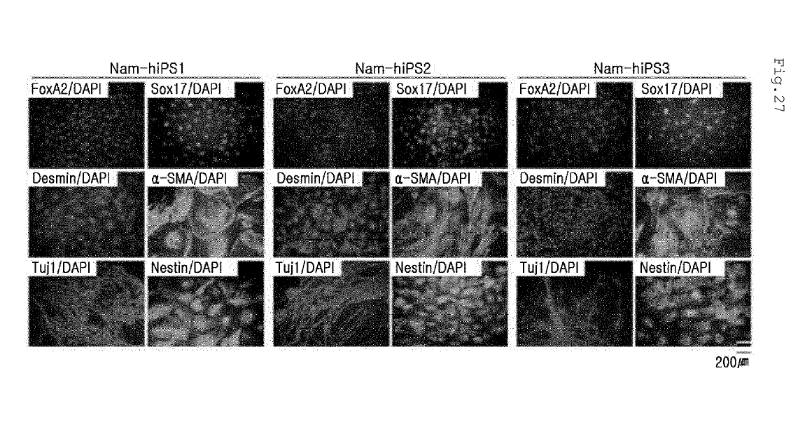

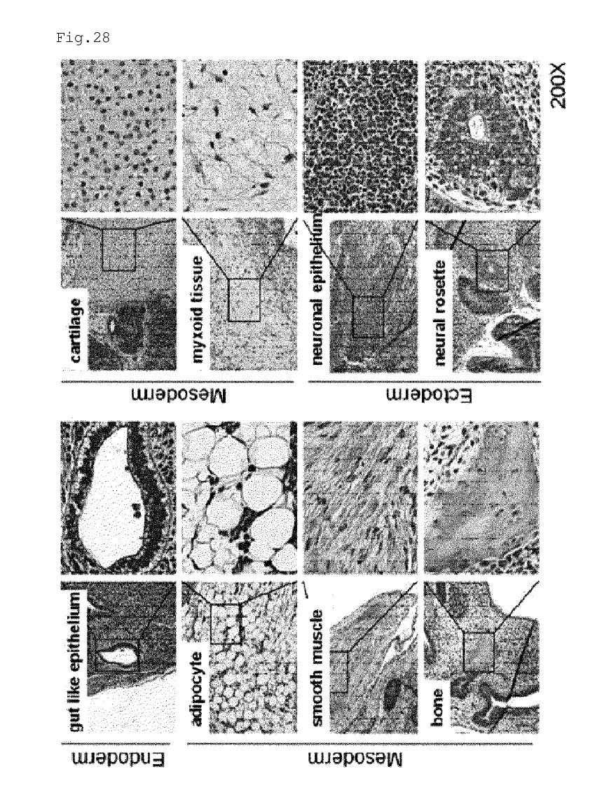

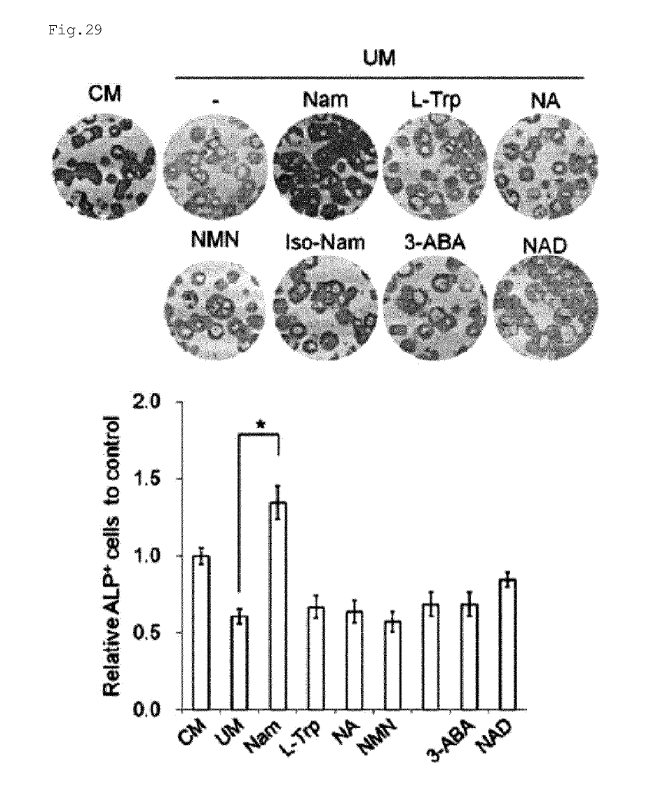

In still another example of the present invention, it was shown that pluripotent stem cells produced in a culture medium treated with nicotinamide maintained hESC-like morphology during a continuous culture process and expressed pluripotency-specific markers at levels similar to those of hESCs (FIGS. 21 and 22) and that four transgenes (OSKM) used in the induction of reprogramming were all inserted into the genome of the host cells (FIG. 23). Moreover, it was verified that the pluripotent stem cells showed the methylation patterns of pluripotency-specific promoters (Oct4 and Nanog) (FIG. 24) and the expression patterns of global genes (FIG. 25) at levels similar to those of hESCs had the ability to differentiate into three germ layers in vitro (FIGS. 26 and 27) and in vivo (FIG. 28).

In another aspect, the present invention provides a method of producing reprogrammed pluripotent stem cells from differentiated cells, the method comprising the steps of: (a) transferring a reprogramming factor to the differentiated cells; and (b) culturing the differentiated cells in a medium containing the composition of the present invention.

Step (a) of transferring the reprogramming factor to the differentiated cells may be performed by any method that is generally used in the art to provide nucleic acid molecules or proteins to cells. Preferably, step (a) may be performed by a method of adding the reprogramming factor to a culture of the differentiated cells, a method of injecting the reprogramming factor directly into the differentiated cells, or a method of infecting the differentiated cells with a virus obtained from packaging cells transfected with a viral vector including a gene of the reprogramming factor.

The method of injecting the reprogramming factor directly into the differentiated cells may be performed using any method known in the art. This method can be suitably selected from among microinjection, electroporation, particle bombardment, direct intramuscular injection, an insulator-based method, and a transposon-based method, but is not limited thereto.

In the present invention, the reprogramming factor can be selected depending on the kind of cell to be reprogrammed. Preferably, the reprogramming factor may be one or more proteins selected from the group consisting of Oct4, Sox2, K1F4, c-Myc, Nanog, Lin-28 and Rex1, or one or more nucleic acid molecules encoding the proteins, but is not limited thereto. More preferably, the reprogramming factor may include Oct4 protein or a nucleic acid molecule encoding the protein and may include Oct4, Sox2, K1F4 and c-Myc proteins or nucleic acid molecules encoding these proteins.

In the present invention, the packaging cells may be selected from among various cells known in the art depending on the kind of viral vector used. Preferably, the packaging cells may be GP2-293 packaging cells, but are not limited thereto.

In addition, the viral vector that is used in the present invention may be selected from among vectors derived from retroviruses, for example, HIV (human immunodeficiency virus), MLV (murine leukemia virus), ASLV (avian sarcoma/leukosis), SNV (spleen necrosis virus), RSV (rous sarcoma virus), MMTV (mouse mammary tumor virus) or the like, lentiviruses, adenovirus, adeno-associated virus, herpes simplex virus, etc, but is not limited thereto. Preferably, the viral vector may be a retroviruse vector. More preferably, it may be the retroviruse vector pMXs.

Steps (a) and (b) may be performed simultaneously, sequentially or in the reverse order. The above-described method may further comprise a step of isolating embryonic stem cell-like colonies from a culture resulting from step (b).

Reprogrammed pluripotent stem cells that are produced by the above-described method may include all in vitro cell cultures obtained by treating differentiated cells with the nicotinamide-containing composition for promoting reprogramming and with the reprogramming factors. The cell culture in the present invention may also include various cells which are being reprogrammed, various proteins, enzymes and transcripts which are obtained during culture of the cells, and culture media containing them.

The differentiated cells and pluripotent stem cells that are used in the present invention are as described above.

The reprogramming of differentiated cells into pluripotent stem cells may correspond to an increase in growth and proliferation of cells, inhibition of apoptosis, an increase in mitochondrial activity, inhibition of senescence, a decrease in oxidative stress, inhibition of p53 signaling, a reduction in reprogramming time or an increase in reprogramming efficiency in reprogrammed cells compared to that in the differentiated cells.

In an example of the present invention, it was shown that reprogrammed pluripotent stem cells, transduced with a reprogramming factor and cultured in nicotinamide-containing medium, showed an increase in growth and proliferation of cells, inhibition of apoptosis, an increase in mitochondrial activity, inhibition of senescence, a decrease in oxidative stress, inhibition of p53 signaling, a reduction in reprogramming time and an increase in reprogramming efficiency compared to those in differentiated cells.

In still another aspect, the present invention provides a medium composition for maintaining or culturing pluripotent stem cells in an undifferentiated state, the medium composition comprising nicotinamide.

In an example of the present invention, cells were cultured in a medium containing nicotinamide, and the total cell number was counted after the culture. As a result, it was shown that the nicotinamide-containing medium had the effect of promoting the growth and proliferation of cells (FIG. 10).

In still another aspect, the present invention provides a method for culturing reprogrammed pluripotent stem cells in an undifferentiated state, the method comprising culturing reprogrammed pluripotent stem cells, produced by the inventive method of producing reprogrammed pluripotent stem cells from differentiated cells, in a medium containing nicotinamide. In other words, according to the present invention, pluripotent stem cells including the reprogrammed pluripotent stem cells are continuously cultured in a nicotinamide-containing medium, thereby providing environmental conditions advantageous for maintaining an undifferentiated state and pluripotency.

As used herein, the term “undifferentiated state” in a broad sense includes a state in which cells have not yet differentiated into specific cell types. Specifically, the term means a state in which the pluripotent stem cells of the present invention no longer differentiate from the original state or are reprogrammed.

In still another aspect, the present invention provides a composition for maintaining pluripotent stem cells in an undifferentiated state, the composition comprising nicotinamide. Preferably, the composition may be a culture medium, and the concentration of nicotinamide in the medium composition may be between 0.01 mM and 20 mM.

In the present invention, the medium composition may further comprise one or more known CDM components, and the addition of nicotinamide can improve the composition and effect of CDM.

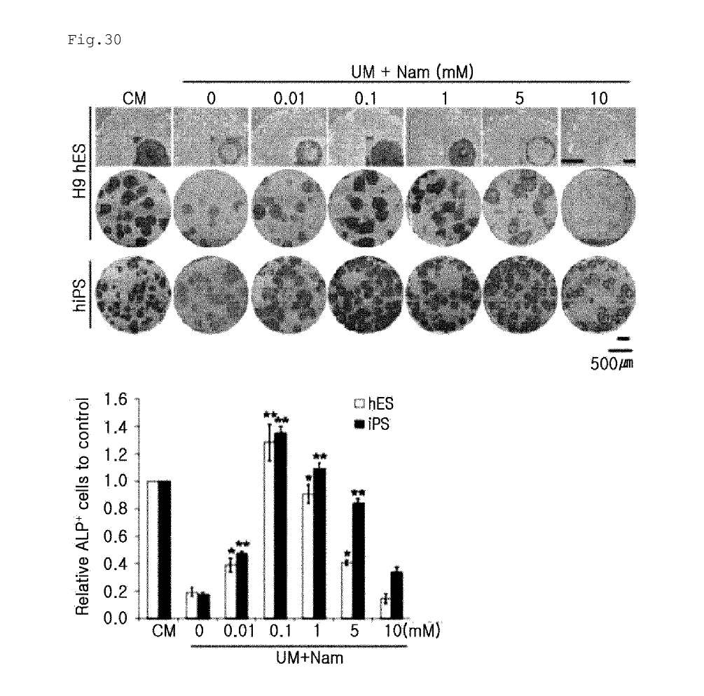

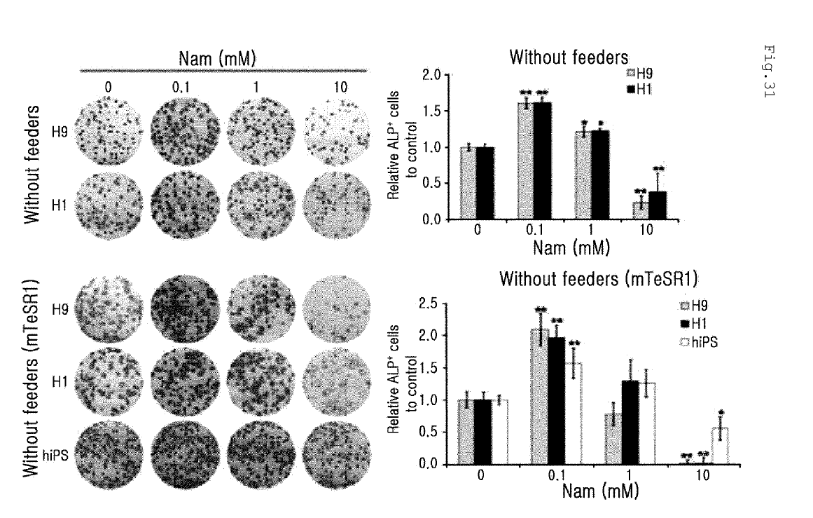

In an example of the present invention, H9 human embryonic stem cells that are typical human pluripotent stem cells were cultured in unconditioned medium (UM) in which a differentiated state is easily induced, and the cells were treated with NAD⁺ precursors (L-tryptophan, Nicotinic acid, NMN, Iso-Nam, and 3-ABA), including nicotinamide, and NAD⁺, and then analysis was performed to examine whether the induction of differentiation could be inhibited and the undifferentiated state could be maintained. As a result, as shown in FIG. 29, the induction of differentiation was most effectively inhibited in the UM medium treated with nicotinamide, and the undifferentiated state was maintained at a level equal to that of cells cultured in conditioned medium (CM) (FIG. 29). In addition, the effect of nicotinamide was observed at various concentrations of nicotinamide, and as a result, it was found that the nicotinamide concentration effective for maintaining the undifferentiated state of human pluripotent stem cells, including human embryonic stem cells and human induced pluripotent stem cells, is in the range from 0.1 to 5 mM (FIG. 30).

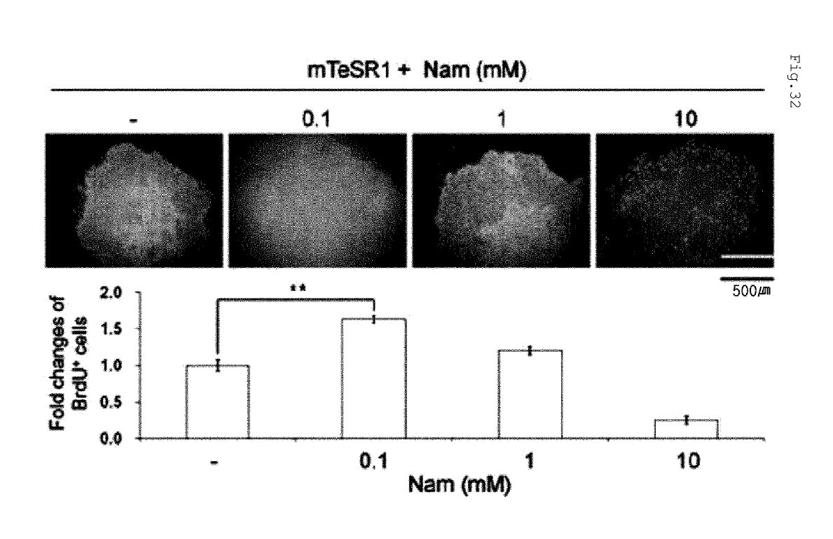

In another example of the present invention, the effects of nicotinamide in two kinds of undifferentiated human embryonic cell lines (H1 and H9) established independently from each other were analyzed. As a result, it was shown that, in medium compositions containing or not containing MEF feeder cells and in chemically-defined culture media free of feeder cells and serum, the addition of nicotinamide at a concentration ranging from 0.1 to 1 mM was effective for maintaining the undifferentiated state (FIG. 31).

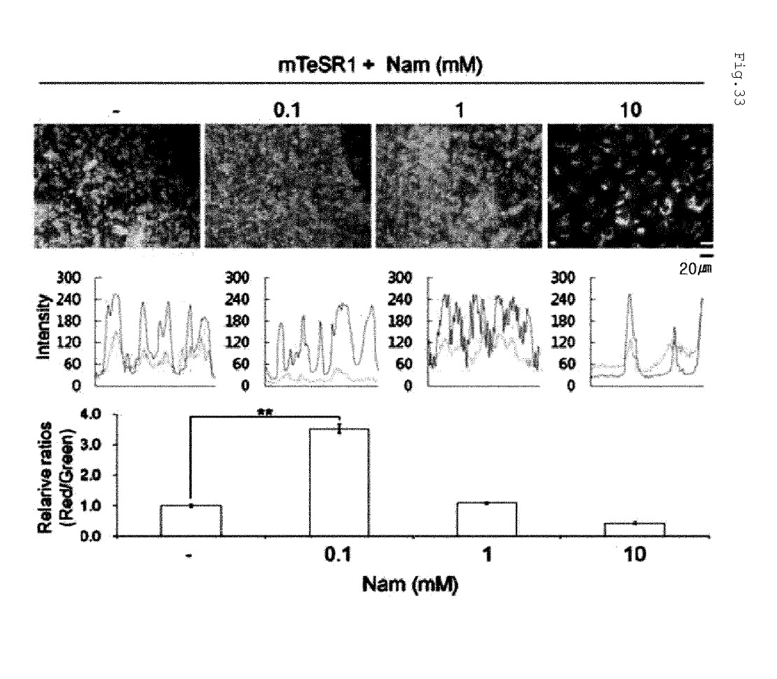

In a still another example of the present invention, in order to examine whether nicotinamide effective for maintaining the undifferentiated state of human pluripotent stem cells also influences the proliferation of human pluripotent stem cells, a BrdU assay was performed. As a result, it was shown that the growth of the cells was significantly increased in a culture medium treated with nicotinamide (FIG. 32).

In the present invention, the concentration of nicotinamide that is used to culture human pluripotent stem cells in an undifferentiated state may preferably be 0.01-20 mM, and more preferably 0.05-10 mM. Most preferably, the concentration of nicotinamide may be 0.1-5 mM (FIGS. 29 to 32).

In still another aspect, the present invention provides a composition for maintaining or improving the mitochondrial function of pluripotent stem cells, the composition comprising nicotinamide.

In the present invention, the mitochondrial function includes the energy production-related metabolic function of mitochondria in cells and is known to be reduced by the cellular senescence process. Particularly, it is known that the mitochondrial function is maintained at a high level in undifferentiated or reprogrammed pluripotent stem cells. Because the mitochondrial function occurs through various ion channels present in the membrane, can be determined by the membrane potential of mitochondria.

In an example of the present invention, the membrane potential (ΔΨm) of mitochondria was measured after treatment with nicotinamide. When human embryonic stem cells were maintained and cultured, the membrane potential of mitochondria in the human embryonic stem cells was measured by JC-1 staining after addition of various concentrations of nicotinamide to mTeSR1. After JC-1 staining, activated mitochondria are stained with red, and non-activated mitochondria are stained with green. The ratio of the number of red-stained mitochondria to the number of green-stained mitochondria is measured, and an increase in the ratio indicates an increase in activated mitochondria. It was shown that the addition of 0.1 mM nicotinamide significantly increased the action potential of mitochondria (FIG. 33). This suggests that the addition of nicotinamide is effective for maintaining the undifferentiated state of pluripotent stem cells by increasing the mitochondrial activity.

In still another aspect, the present invention provides a method of culturing pluripotent stem cells so as to be maintained in an undifferentiated state, the method comprising culturing the cells using the composition for maintaining the undifferentiated state of pluripotent stem cells, which comprises nicotinamide.

In still another aspect, the present invention provides a method for preparing a cell culture, the method comprising culturing pluripotent stem cells so as to be maintained in an undifferentiated state using the composition for maintaining the undifferentiated state of pluripotent stem cells, which comprise nicotinamide.

The method of culturing pluripotent stem cells so as to be maintained in an undifferentiated state and the method of preparing the cell culture enables the pluripotent stem cells to be maintained in an undifferentiated state in the presence or absence of animal serum or feeder cells. The culture may be a plurality of continuous subcultures.

The pluripotent stem cells, undifferentiated state and cell culture of the present invention are as described above.

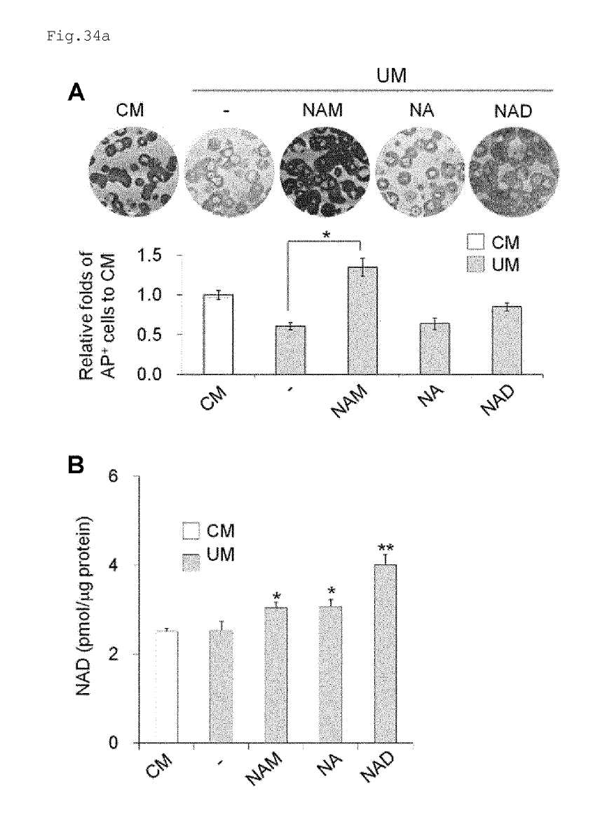

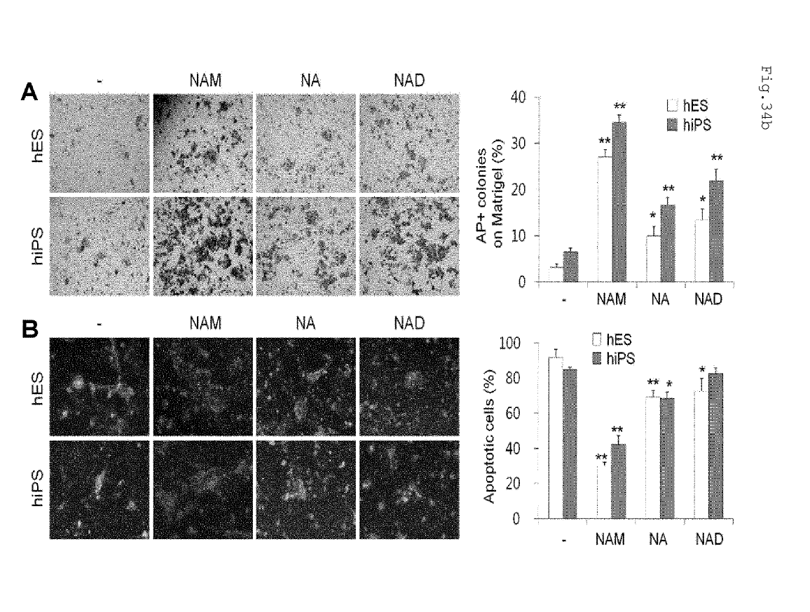

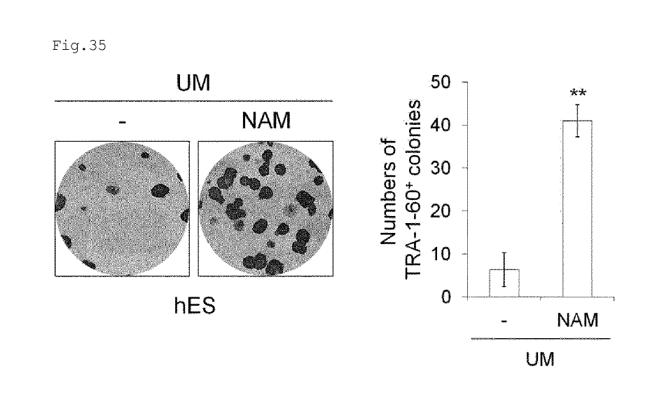

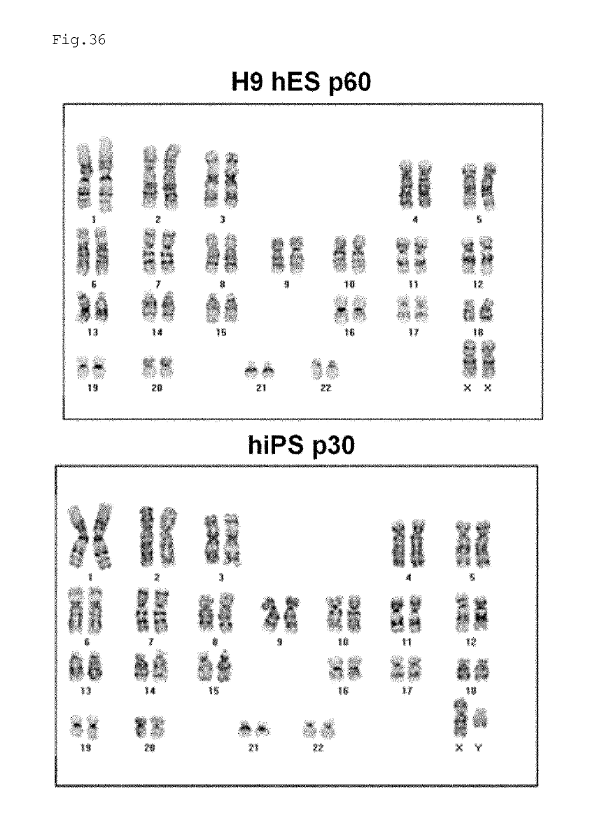

Generally, H9 human pluripotent stem cells generally differentiate in UM culture medium. However, it was shown that, when 0.1 mM of nicotinamide was added, H9 human pluripotent stem cells did not differentiate and could be maintained in an undifferentiated state (FIG. 34A). In addition, the results of AP staining and apoptosis assays indicated that the addition of nicotinamide significantly increased the survival and self-renewal abilities of human pluripotent stem cells, including H9 human embryonic stem cells and human induced pluripotent stem cells, even when the cells were cultured at low density after single-cell dissociation (FIG. 34B). Further, the results of TRA-1-60 staining indicated that, even in differentiation media such as UM, the addition of 0.1 mM nicotinamide enabled stem cells to be subcultured for a long period of time for 10 or more passages in an undifferentiated state (FIG. 35). Also, normal karyotypes could be observed even after the long-term subculture (FIG. 36). Such results indicate that nicotinamide can control cellular and molecular mechanisms to improve conditions for maintaining and culturing pluripotent stem cells in an undifferentiated state.

In still another aspect, the present invention provides a cell culture comprising: pluripotent stem cells; and a composition for improving the mitochondrial function of pluripotent stem cells, which comprises nicotinamide.

The present invention encompasses all in vitro cell cultures that are obtained by treating pluripotent stem cells with a composition for improving the mitochondrial function of pluripotent stem cells, which comprises nicotinamide. The cell culture according to the present invention may also include various cells which are being cultured, various proteins, enzymes and transcripts which are obtained during culture of the cells, and a culture medium containing them.

The pluripotent stem cells of the present invention are as described above.

In still another aspect, the present invention provides a method for establishing an embryonic stem cell line capable of being maintained in an undifferentiated state, the method comprising the steps of: obtaining embryonic stem cells; and culturing the embryonic stem cells under culture conditions including the medium composition to obtain the embryonic stem cell line.

Herein, the embryonic stem cells include embryonic stem cells derived from any animals, including humans, monkeys, pigs, horses, cattle, sheep, dogs, cats, mice, rabbits and the like. Preferably, the embryonic stem cells are embryonic stem cells of human origin.

### MODE FOR INVENTION

Hereinafter, the present invention will be described in further detail with reference to examples. It is to be understood, however, that these examples are for illustrative purposes only and are not intended to limit the scope of the present invention.

### Example 1

### Culture of Human Embryonic Stem Cells and Induced Pluripotent Stem Cells

Human embryonic stem cells (hESC) H9 (NIH Code, WA09; WiCell Research Institute, Madison, Wis.) and H1 (NIH Code, WA01; WiCell Research Institute) and induced pluripotent stem cells (hiPSC) were each cultured with hESC culture medium (unconditioned medium; UM) or MEF-CM (conditioned medium) on γ-irradiated MEFs (mouse embryonic fibroblasts) or on plates coated with Matrigel (BD Biosciences, Franklin Lakes, N.J.). The cultured human embryonic stem cells and induced pluripotent stem cells were treated with collagenase IV (1 mg/ml; Invitrogen) or dispase (1 mg/ml; Invitrogen) once a week and subcultured. MEF-CM was prepared as γ-irradiated MEF according to a known method (Xu C. Nat Biotechnol 19, 971-974) and supplemented with 8 ng/ml of bFGF. UM contained 80% DMEM/F12, 20% knockout serum replacement (KSR, Invitrogen, Carlsbad, Calif.), 1% non-essential amino acids (NEAA, Invitrogen), 1 mM L-glutamine (Invitrogen), 0.1 mM β-mercaptoethanol (Sigma, St. Louis, Mo.) and 6 ng/ml bFGF (basic fibroblast growth factor, Invitrogen). Particularly, for culture in the absence of feeder cells and serum, human embryonic stem cells and human induced pluripotent stem cells were cultured in mTeSR1 medium (StemCell Technologies). Human newborn foreskin fibroblasts (hFF, ATCC, catalog number CRL-2097; American Type Culture Collection, Manassas, Va.) were cultured in a DMEM medium containing 10% FBS (fetal bovine serum, Invitrogen), 1% non-essential amino acids, 1 mM L-glutamine and 0.1 mM β-mercaptoethanol.

### Example 2

### Production of Retrovirus and Induction of hiPSCs

A pMXs vector comprising the human cDNA of OCT4 (POU5F1), SOX2, c-MYC (MYC) and K1F4, as disclosed in Takahashi, K. et al. Cell 131, 2007, 861-872, was purchased from Addgene. GP2-293 packaging cells were transfected with a retroviral vector DNA and a VSV-G envelop vector using Lipofectamine 2000. At 24 hours after the transfection, the supernatant containing the first virus was collected, and then the medium was replaced, and after 24 hours, the supernatant containing the second virus was collected. The supernatant was sterilized through a filter having a pore size of 0.45 μm, after which it was centrifuged at 20,000 rpm for 90 minutes and stored at −70° C. until use.

For production of iPSC, human foreskin fibroblasts (hFFs) were seeded on gelatin-coated 6-well plates at a concentration of 1×10⁵ cells per well at 6 hours before transfection and were transfected with virus in the presence of polybrene (6 μg/ml). At 5 days after the transfection, the hFFs were collected by trypsin treatment and reseeded on Matrigel-coated 6-well plates at a concentration of 5-6×10⁴ cells per well in order to perform experiments in feeder-free conditions. The medium was replaced with MEF-CM medium containing 10 ng/ml of bFGF. The medium was replaced at 2-day intervals. At 20 days after the transfection, hESC-like colonies were collected and transferred to 12-well plates having MEFs as feeder cells, and then the colonies were continuously cultured using the hESC culture method described in Example 1.

In order to measure the efficiency of reprogramming into human induced pluripotent stem cells, the number of colonies stained with the embryonic stem cell marker ALP on the Matrigel-coated 6-well plate was counted, and the efficiency of reprogramming was calculated. Each experiment was performed in triplicate.

### Example 3

### Microarray Assay

Total RNA was isolated from an induced pluripotent stem cell line (Nam-iPS) induced from human fibroblasts, H9 human embryonic stem cells (hESs) and human fibroblasts (hFFs). The isolated total RNA was extracted using an RNA Mini kit (Qiagen) and labeled with Cy3 and hybridized onto the Agilent human whole genome 4X44K microarray (based on single color) according to the manufacturer's instruction. The hybridized images were scanned using Agilent's DNA microarray scanner and quantified with Feature Extraction software (Agilent Technology, Palo Alto, Calif.). All data normalization and selection of fold-changed genes were performed using GeneSpringGX 7.3 (Agilent Technology, USA). The averages of normalized ratios were calculated by dividing the average of normalized signal channel intensity by the average of normalized control channel intensity. Functional annotation of genes was performed according to Gene Ontology™ Consortium by selected gene using GeneSpringGX 7.3 (http://www.geneontology.org/index.shtml), and Gene classification was based on searches done by BioCarta (http://www.biocarta.com/), GenMAPP (http://www.genmapp.org/), DAVID (http://david.abcc.ncifcrf.gov/), and Medline databases (http://www.ncbi.nlm.nih.gov/).

### Example 4

### RNA Extraction, Reverse Transcription and PCR Analysis

Total RNA was isolated from produced cells using an RNeasy Mini kit (Qiagen, Valencia, Calif.) and reverse-transcribed using a SuperScript First-strand synthesis system kit (Invitrogen) according to the manufacturer's instruction. Then, semi-quantitative RT-PCR was performed using a platinum Tag SuperMix kit (Invitrogen) under the following conditions: 94° C. for 3 minutes, and then 25-30 cycles, each consisting of 94° C. for 30 sec, 60° C. for 30 sec and 72° C. for 30 sec, followed by extension at 72° C. for 10 minutes. The sequences of the primers used in the RT-PCR are shown in Table 1 below.

TABLE 1

| Gene | Primer (Forward) | Primer (Reverse) | Accession No. |
|---|---|---|---|
| Total OCT4 | GAGAAGGATGTGGTCCGAGTGTG | CAGAGGAAAGGACACTGGTCCC | NM_002701 |
| | (SEQ ID NO: 1) | (SEQ ID NO: 2) | |
| Total SOX2 | AGAACCCCAAGATGCACAAC | ATGTAGGTCTGCGAGCTGGT | NM_003106 |
| | (SEQ ID NO: 3) | (SEQ ID NO: 4) | |
| Total KLF4 | ACCCTGGGTCTTGAGGAAGT | ACGATCGTCTTCCCCTCTTT | |
| | (SEQ ID NO: 5) | (SEQ ID NO: 6) | |
| Total c-Myc | CCTACCCTCTCAACGACAGC | CTCTGACCTTTTGCCAGGAG | NM_002467 |
| | (SEQ ID NO: 7) | (SEQ ID NO: 8) | |
| Endo OCT4 | GACAGGGGGAGGGGAGGAGCTAGG | CTTCCCTCCAACCAGTTGCCCCAAAC | |
| | (SEQ ID NO: 9) | (SEQ ID NO: 10) | |
| Endo SOX2 | GGGAAATGGGAGGGGTGCAAAAGAGG | TTGCGTGAGTGTGGATGGGATTGGTG | |
| | (SEQ ID NO: 11) | (SEQ ID NO: 12) | |
| Endo KLF4 | AGCCTAAATGATGGTGCTTGGT | TTGAAAACTTTGGCTTCCTTGTT | |
| | (SEQ ID NO: 13) | (SEQ ID NO: 14) | |
| Endo c-Myc | CGGGCGGGCACTTTG | GGAGAGTCGCGTCCTTGCT | |
| | (SEQ ID NO: 15) | (SEQ ID NO: 16) | |
| hTERT | CGGAAGAGTGTCTGGAGCAA | GGATGAAGCGGAGTCTGGA | NM_198255.1 |
| | (SEQ ID NO: 17) | (SEQ ID NO: 18) | |

For transgene and genomic integration

| | | |
|---|---|---|
| Trans OCT4 | GAGAAGGATGTGGTCCGAGTGTG | CCCTTTTTCTGGAGACTAAATAAA |
| | (SEQ ID NO: 19) | (SEQ ID NO: 20) |
| Trans SOX2 | GGCACCCCTGGCATGGCTCTTGGCTC | TTATCGTCGACCACTGTGCTGCTG |
| | (SEQ ID NO: 21) | (SEQ ID NO: 22) |
| Trans KLF4 | ACGATCGTGGCCCCGGAAAAGGACC | TTATCGTCGACCACTGTGCTGCTG |
| | (SEQ ID NO: 23) | (SEQ ID NO: 24) |
| Trans c-Myc | CAACAACCGAAAATGCACCAGCCCCAG | TTATCGTCGACCACTGTGCTGCTG |
| | (SEQ ID NO: 25) | (SEQ ID NO: 26) |

hESC markers

| | | | |
|---|---|---|---|
| NANOG | CAAAGGCAAACAACCCACTT | ATTGTTCCAGGTCTGGTTGC | NM_024865 |
| | (SEQ ID NO: 27) | (SEQ ID NO: 28) | |

Ectoderm lineage markers

| | | | |
|---|---|---|---|
| NCAM | AGGAGACAGAAACGAAGCCA | GGTGTTGGAAATGCTCTGGT | NM_000615 |
| | (SEQ ID NO: 29) | (SEQ ID NO: 30) | |
| PAX6 | GCCAGCAACACACCTAGTCA | TGTGAGGGCTGTGTCTGTTC | NM_000280 |
| | (SEQ ID NO: 31) | (SEQ ID NO: 32) | |
| VIM | gggacctctacgaggaggag | cgcattgtcaacatcctgtc | NM_003380 |
| | (SEQ ID NO: 33) | (SEQ ID NO: 34) | |
| OTX1 | TAACCCTACGCCCTCCTCTTCCTACT | AAGCAGTCGGCAGAGTTGAAGGCAAG | NM_014562 |
| | (SEQ ID NO: 35) | (SEQ ID NO: 36) | |

Mesoderm lineage markers

| | | | |
|---|---|---|---|
| cTnT | GGCAGCGGAAGAGGATGCTGAA | GAGGCACCAAGTTGGGCATGAAC | NM_000364 |
| | (SEQ ID NO: 37) | (SEQ ID NO: 38) | |
| IGF2 | CAGACCCCCAAATTATCGTG | GCCAAGAAGGTGAGAAGCAC | NM_000612 |
| | (SEQ ID NO: 39) | (SEQ ID NO: 40) | |
| TBX6 | TACATTCACCCCGACTCTCC | TGTATGCGGGGTTGGTACTT | NM_004608 |
| | (SEQ ID NO: 41) | (SEQ ID NO: 42) | |
| RUNX2 | cactcactaccacacctacc | gtcgccaaacagattcatcc | NM_001024630 |
| | (SEQ ID NO: 43) | (SEQ ID NO: 44) | |

Endoderm lineage markers

| | | | |
|---|---|---|---|
| HGF | gcatcaaatgtcagccctgg | caacgctgacatggaattcc | NM_000601 |
| | (SEQ ID NO: 45) | (SEQ ID NO: 46) | |
| AMYLASE | GCTGGGCTCAGTATTCCCCAAAT | GACGACAATCTCTGACCTGAGTAG | NM_000699 |
| | (SEQ ID NO: 47) | (SEQ ID NO: 48) | |
| HAND1 | tgcctgagaaagagaaccag | atggcaggatgaacaaacac | NM_004821 |
| | (SEQ ID NO: 49) | (SEQ ID NO: 50) | |
| PECAM | ccacatacactccttccacc | gactacccaaaactacaagcc | NM_000442 |
| | (SEQ ID NO: 51) | (SEQ ID NO: 52) | |
| GAPDH | GAAGGTGAAGGTCGGAGTC | GAAGATGGTGATGGGATTTC | NM_002046 |
| | (SEQ ID NO: 53) | (SEQ ID NO: 54) | |

For bisulfate sequencing

| | | |
|---|---|---|
| OCT4-1 | ATTTGTTTTTTGGGTAGTTAAAGGT | CCAACTATCTTCATCTTAATAACATCC |
| | (SEQ ID NO: 55) | (SEQ ID NO: 56) |
| OCT4-2 | GGATGTTATTAAGATGAAGATAGTTGG | CCTAAACTCCCCTTCAAAATCTATT |
| | (SEQ ID NO: 57) | (SEQ ID NO: 58) |
| NANOG | TGGTTAGGTTGGTTTTAAATTTTTG | AACCCACCCTTATAAATTCTCAATTA |
| | (SEQ ID NO: 59) | (SEQ ID NO: 60) |

### Example 5

### Measurement of NAD

Embryonic stem cells were treated with nicotinamide and related compounds, and after 6 days, protein was isolated from the cells. NAD cycling enzyme was added to and reacted with 20 μg of the protein per each group at room temperature for 5 minutes, and then a NADH developer was added and reacted therewith for 2-3 hours. The absorbance at 450 nm was measured using a microplate reader, and the amount of the protein was quantified.

### Example 6

### Alkaline Phosphatase (AP) Staining

AP staining was performed using a commercial AP kit (Sigma) according to the manufacturer's instruction. Images of AP-positive cells were recorded with HP Scanjet G4010. Also, bight field images were obtained with an Olympus microscope (IX51, Olympus, Japan).

### Example 7

### Dual Apoptosis Analysis

In order to examine the effect of nicotinamide on a reduction in the apoptosis of human pluripotent stem cells, dual apoptosis assay (Biotium, Hayward, Calif.) was performed. Embryonic stem cells or cells reprogrammed were stained with NucView™488 Caspase-3 Substrate (green)/Annextin V (red) at room temperature for 30 minutes, followed by washing with PBS. At least 4 sections of each sample were imaged with an Olympus fluorescence microscope (IX51, Olympus), and the number of cells stained with green (Caspase-3) was counted to quantify the degree of apoptosis.

### Example 8

### Immunocytochemistry

For immunostaining, cells were seeded on Matrigel-coated 4-well Lab-Tek chamber slides (Nunc, Naperville, Ill.) and cultured for 5 days under the indicated conditions. The cells were fixed in 4% paraformaldehyde at room temperature for 15 minutes, and then washed with PBS/0.2% BSA. Next, the cells were passed through PBS/0.2% BSA/0.1% Triton X-100 for 15 minutes, and then incubated with 4% normal donkey serum (Molecular Probes, Eugene, Oreg., USA) in PBS/0.2% BSA at room temperature for 1 hour. The cells were diluted with PBS/0.2% BSA, and then reacted with primary antibody at 4° C. for 2 hours. After washing, the cells were reacted with FITC- or Alexa594-conjugated secondary antibody (Invitrogen) in PBS/0.2% BSA at room temperature for 1 hour. The cells were counter-stained with 10 μg/ml DAPI. The chamber slide was observed with an Olympus microscope or an Axiovert 200M microscope (Carl Zeiss, Gottingen, Germany). The antibodies used in this Example are shown in Table 2 below.

TABLE 2

| Antibodies | Catalog No. | Company | Dilution |
|---|---|---|---|
| anti-p16 | 4824 | Cell Signaling Technology | 1:1000 |
| anti-p-p53 | 9286 | Cell Signaling Technology | 1:500 |
| (Ser 15) | | | |
| anti-p-p53 | 2528 | Cell Signaling Technology | 1:250 |
| (Ser 315) | | | |
| anti-p53 | sc-126 | Santa Cruz Biotechnology | 1:2000 |
| anti-p21 | sc-397 | Santa Cruz Biotechnology | 1:4000 |
| anti-p27 | 2552 | Cell Signaling Technology | 1:1000 |
| anti-p-ERK | 9101 | Cell Signaling Technology | 1:1000 |
| anti-Cytochrome c | 4272 | Cell Signaling Technology | 1:1000 |
| anti-PARP | 9532 | Cell Signaling Technology | 1:1000 |
| anti--actin | 5316 | Sigma | 1:0000 |
| anti-OCT4 | sc-9081 | Santa Cruz Biotechnology | 1:50 |
| anti-NANOG | sc-33759 | Santa Cruz Biotechnology | 1:200 |
| anti-NANOG | AF1997 | R&D Systems | 1:100 |
| anti-SSEA-3 | MAB1434 | R&D Systems | 1:50 |
| anti-SSEA-4 | MAB1435 | R&D Systems | 1:50 |
| anti-TRA-1-60 | MAB4360 | Chemicon | 1:100 |
| anti-TRA-1-81 | MAB4381 | Chemicon | 1:100 |
| In vitro | | | |
| differentiation | | | |
| anti-TUJ1 | PRB-435P | Covance | 1:500 |
| anti-NESTIN | MAB5326 | Chemicon | 1:100 |
| anti-FOXA2 | 07-633 | Millipore | 1:100 |
| anti-SOX17 | MAB1924 | R&D | 1:50 |
| anti--SMA | A5228 | Sigma | 1:400 |
| anti-DESMIN | AB907 | Chemicon | 1:30 |

### Example 9

### Chromatin Immunoprecipitation Assay

Formaldehyde was added to a cell culture to fix the cells, and then the chromatin was fragmented to a suitable size by sonication. The chromatin was immunoprecipitated with antibodies to lysine residues 4 and 27 of histone 3, and then the DNA was collected and amplified by real-time polymerase chain reaction using primers for the Oct4 and Nanog promoter regions. The antibodies and primers used in this Example are shown in Table 3 below.

TABLE 3
For ChIP

| Antibodies | Catalog No. | Company | Dilution |
|---|---|---|---|
| Anti-dimethyl-H3 | 07-030 | Millipore | 1:200 |
| (Lys4) | | | |
| Anti -trimethyl- | 07-449 | Millipore | 1:200 |
| H3 (Lys27) | | | |

| Gene | Primer(Forward) | Primer(Reverse) |
|---|---|---|
| OCT4 | TTGCCAGCCATTATCATTCA | TATAGAGCTGCTGCGGGATT |
| | (SEQ ID NO: 61) | (SEQ ID NO: 62) |
| NANOG | GATTTGTGGGCCTGAAGAAA | GGAAAAAGGGGTTTCCAGAG |
| | (SEQ ID NO: 63) | (SEQ ID NO: 64) |

### Example 10

### Growth Efficiency Test

Human fibroblasts were seeded in a 6-well culture dish at a density of about 1×10⁵ cells per well. The seeded cells were cultured for 26 days under the indicated culture conditions. To count the cell number, the cells were washed with PBS and treated with trypsin. The cell suspension was mixed with 0.4% (wt/vol) trypan blue solution, and the number of viable cells was counted on day 19 and day 26 using a hemocytometer (FIG. 10). Each experiment was performed in triplicate.

### Example 11

### BrdU Incorporation

For 5-bromo)-2-deoxyuridine (BrdU; BD Pharmingen, San Diego) incorporation assays, human embryonic stem cells were cultured on Matrigel-coated 4-well LabTec chamber slides for 4 days.

Specifically, for BrdU incorporation, cells were incubated in the presence of 30 μM BrdU at 37° C. for 1 hour. After washing with PBS, the cells were fixed with 4% paraformaldehyde for 15 minutes and reacted in 1N HCl at room temperature for 15 minutes. The sample was washed and reacted with 0.1 M sodium tetraborate for 15 minutes. After washing, the cells were reacted with anti-BrdU antibody in 3% BSA-containing PBS for 1 hour, and then reacted with FITC-conjugated secondary antibody for 30 minutes. The nuclei were counter-stained with DAPI, and then the cells were observed with an Olympus microscope.

### Example 12

### Live-Cell Imaging Cell Cycle Analysis

On day 12 of induction of reprogramming, virus expressing the cell-cycle regulators Geminin-GFP (green) and Cdt1-RFP (red) was introduced into the cells for 2 hours while slowly shaking the cells, and then an accelerator was added to the cells, followed by culture for 1 hour. The medium was replaced with fresh medium, and the cells were further cultured overnight and imaged with a fluorescence microscope. The images were obtained for at least 4 sections per sample, and the number of resting-stage cells (red) and dividing cells (green) was counted. The ratio of dividing cells was quantified by the green/red ratio.

### Example 13

### Senescence-Associated β-Galactosidase (SA-β-Gal) Staining and Senescence-Associated DNA Damage Staining

On day 26 of induction of reprogramming, the cells were fixed with a fixation solution at room temperature for 10 minutes, and then stained with a β-galactosidase solution at room temperature for 15 minutes. Next, the DNA was stained with DAPI (4′,6-diamidino-2-phenylindole) for 5 minutes, and then the cells were imaged by phase-contrast and fluorescence microscopy. The number of cells having senescence (green) damaged DNA (blue combined points) was counted and quantified relative to the number of normal cells.

### Example 14

### Measurement of Reactive Oxygen Species (ROS)

On day 19 of induction of reprogramming, the cells were dissociated into single cells by trypsin, and then stained with 5 μM of CM-H₂DCFDA (2,7-dichlorodihydrofluresceindiacetat) at 37° C. for 30 minutes. Cells treated with 100 μM of hydrogen peroxide (H₂O₂) were used as a positive control group for the formation of reactive oxygen species, and the degree of fluorescence staining was measured by flow cytometry and quantified.

### Example 15

### Quantification of Protein Damage Caused by Oxidative Stress

On days 12, 19 and 26 of induction of reprogramming, protein was isolated from a control group and a nicotinamide-treated group (Nam), after which it was quantified and treated with 2,4-dinitrophenylhydrazine to expose the carbonyl group. The protein was separated according to molecular weight using 10% SDS-polyacrylamide gel, and then protein damage caused by oxidative stress was analyzed using dinitrophenyl antibody. After normalization to the expression level of β-actin, the degree of damage was quantified relative to the degree of damage to hFFs taken as 1.

### Example 16

### Measurement of Mitochondrial Membrane Potential

At different time points of reprogramming, the cells of a control group and a nicotinamide-treated group (Nam) were dissociated with single cells by trypsin, and then stained with JC-1 (5,5′,6,6′-tetraachloro-1,1′,3,3′-tetraethylbenzimidazolocarbocyanine iodide) solution at 37° C. for 15 minutes. After washing, the degree of fluorescence staining was measured by flow cytometry, relative mitochondrial membrane potential was quantified by the red/green ratio.

### Example 17

### Western Blot Analysis

Cells were lysed with RIPA buffer (containing 50 mM Tris, pH 8.0, 150 mM NaCl, 1% NP-40, 0.1% SDS, 0.5% deoxycholic acid, 1 mM PMSF) and mixed with a protease inhibitor (Roche Applied Science, Indianapolis, Ind.) on ice for 15 minutes, followed by centrifugation at 20,000×g at 4° C. for 10 minutes. The supernatant was re-centrifuged for 10 minutes, and the protein concentration was measured using a BCA protein assay kit (Pierce, Rockford, Ill.). The protein (20 μg) was separated by SDS-PAGE (polyacrylamide gel electrophoresis) and adsorbed onto a PVDF (polyvinylidene fluoride) membrane (Millipore Corp, Bedford, Mass.). The membrane was reacted in PBS containing 0.1% Tween-20 and 5% non-fat milk at room temperature for 2 hours and was probed for 1 hour with primary antibody diluted with PBS/0.2% BSA. After washing, the membrane was reacted with HRP-conjugated anti-rabbit or HRP-conjugated anti-mouse secondary antibody (Amersham, Arlington Heights, Ill.), and the bands were visualized with ECL Advance kit (Amersham). The density of the bands was analyzed using Image Gauge software (Fuji Photo Film GMBH, D) and normalized to the bands of the loading control (β-actin). Each experiment was performed in triplicate. The error bar indicates the standard error of the mean (n=3). The antibodies used in this Example are shown in Table 2 above.

### Example 18

### Embryoid Body Differentiation

In order to measure the potential of hESC differentiation, human embryoid bodies (hEBs) were cultured in hEB medium (DMEM/F12 containing 10% serum replacement) in non-tissue culture treated Petri dishes. After 5 days of growth in suspension, the embryoid bodies were transferred to gelatin-coated plates and cultured in hEB medium. The cells attached to the bottom of the plate were allowed to stand under the above-described conditions for 15 days so as to differentiate while replacing the medium, if necessary.

### Example 19

### Analysis of Promoter Methylation of Reprogramming Transcription Factors

In order to verify the characteristics of human embryonic stem cells and induced pluripotent stem cells established using gene-transfected retrovirus, promoter methylation of Oct3/4 and Nanog that are human embryonic stem cell-specific transcription factors was analyzed. To extract genomic DNA, reprogrammed stem cells and human embryonic stem cells, cultured in human embryonic stem cell media for 6 days, were extracted using a DNA extraction kit (Qiagen Genomic DNA purification kit). Bisulfite sequencing was performed in three steps. In the first step, DNA was modified using sodium bisulfite, and in the second step, the gene region (generally promoter region) to be analyzed was amplified by PCR, and in the third step, the PCR product was sequenced to determine the degree of methylation of DNA. The DNA modification process using sodium bisulfite was performed using commercial EZ DNA Methylation Kit (Zymo Research). When DNA is treated with bisulfite, methylated cytosine does not change, whereas unmethylated cytosine is converted into uracil. Thus, when DNA is amplified by PCR using primers specific for the nucleotide sequences of cytosine and uracil, methylated DNA and unmethylated DNA can be distinguished from each other. The primers used are shown in Table 4 below.

TABLE 4

| | | | |
|---|---|---|---|
| | Primer | Primer | Accession |
| Gene | (Forward) | (Reverse) | No. |

For bisulfate sequencing

| | | | |
|---|---|---|---|
| bi Oct4-1 | ATTTGTTTTTTGGG | CCAACTATCTTCATC | NM_002701 |
| | TAGTTAAAGGT | TTAATAACATCC | |
| | (SEQ ID NO: 55) | (SEQ ID NO: 56) | |
| bi Oct4-2 | GGATGTTATTAAGA | CCTAAACTCCCCTTC | NM_002701 |
| | TGAAGATAGTTGG | AAAATCTATT | |
| | (SEQ ID NO: 57) | (SEQ ID NO: 58) | |
| bi Nanog | TGGTTAGGTTGGTT | AACCCACCCTTATAA | NM_024865 |
| | TTAAATTTTTG | ATTCTCAATTA | |
| | (SEQ ID NO: 59) | (SEQ ID NO: 60) | |

The PCR reaction mix consisted of 1 μg bisulfite-treated DNA, 0.25 mM/l dNTP, 1.5 mM/l MgCl₂, 50 pM primer, 1×PCR buffer and 2.5 U Platinum Taq DNA polymerase (Invitrogen, Carlsbad, Calif., USA) and had a final volume of 20 μl. The PCR reaction was performed under the following conditions: initial denaturation at 95° C. for 10 min, and then 40 cycles, each consisting of 95° C. for 1 min, 60° C. for 1 min and 72° C. for 1 min, followed by final extension at 72° C. for 10 min. The PCR reaction product was electrophoresed on 1.5% agarose gel, and after gel electrophoresis, it was cloned into a pCR2.1-TOPO vector (Invitrogen). The nucleotide sequences of methylated and unmethylated DNAs were analyzed by sequencing using a M13 primer pair.

### Example 20

### Analysis of Characteristics of Human Induced Pluripotent Stem Cells Induced by Nicotinamide

20-1. Analysis of Expression of Human Embryonic Stem Cell Markers

The stem cell characteristics of the reprogrammed stem cell lines (Nam-iPS) induced from human skin fibroblasts by the addition of nicotinamide were analyzed by ALP staining and immunostaining. Three cell lines (Nam-iPS1, Nam-iPS2 and Nam-iPS3) were analyzed. As a result, the Nam-iPS cell lines were very similar such that they were not distinguished morphologically or by the ALP staining and immunostaining of human embryonic stem cell markers (OCT4, NANOG, SSEA-3, SSEA-4, TRA-1-60, and TRA-1-81) (FIG. 21). In addition, the mRNA expression levels of Oct4, Sox2, c-Myc and Klf4 were analyzed by semi-quantitative RT-PCR using the primer in Table 1, and as a result, it was shown that the Nam-iPS cell lines expressed Oct4, Sox2, cMyc and Klf4 at the total and endogenous levels similar to those in human embryonic stem cells and that the silencing of the genes introduced by retroviruses was completely completed (FIG. 22).

20-2: Analysis of Genomic Integration of Pluripotent Stem Cells Induced by Nicotinamide

Genomic integration of each of Nam-iPS1, Nam-iPS2 and Nam-iPS3 was analyzed. Specifically, genomic DNA was extracted from each of the cell lines using a DNeasy kit (Qiagen, Valencia, Calif.), and 300 ng of each of the genomic DNAs was amplified by PCR using primers (Table 1) capable of specifically amplifying the transferred gene. As a result, it was found that, in the Nam-iPS cell lines, Oct4, Sox2, c-Myc and Klf4 were integrated (FIG. 23). Herein, the human embryonic stem cell line (H9, hES) and the human skin fibroblast cell line (CRL2097, hFF) were used as control groups.

20-3: Analysis of Methylation in Pluripotent Stem Cells Induced by Nicotinamide

According to the method described in Example 19, the methylation degrees of the promoter regions of the stem cell markers Oct4 and Nanog genes in the Nam-iPS cell lines were analyzed by bisulfite sequencing. As a result, as can be seen in FIG. 24, the Nam-iPS-induced pluripotent stem cell lines showed demethylation patterns similar to those of the human embryonic stem cell line (H9), but the parent cells (hFFs) still maintained methylation (FIG. 24).

20-4: Analysis of Gene Profiles of Pluripotent Stem Cells Induced by Nicotinamide

In order to examine the gene expression profiles of the pluripotent stem cell lines (Nam-iPS) induced from human fibroblasts by the addition of nicotinamide, H9 human embryonic stem cells (hESs) and human fibroblasts (hFFs), each of the samples prepared by the method described in Example 3 was analyzed by microarray assay. Changes in gene expression were analyzed based on fold-change and 2-dimensional hierarchical clustering. As can be seen in the hierarchical sample tree in FIG. 25, as expected by the present inventors, the correlation between hES and Nam-iPS was high, whereas the correlation between hFF and Nam-iPS was relatively low. Such results suggest that, when human fibroblasts are reprogrammed into induced pluripotent stem cells by nicotinamide, the expression of genes therein are regulated, similar to those in human embryonic stem cells. Also, the scatter plots showing a comparison of gene expression profiles between Nam-iPS, hES and hFF were comparatively analyzed, and as a result, it could be seen that the expression patterns of OCT4, SOX2, c-Myc, KLF4, NANOG, LIN28 and REX1 were similar between hES and Nam-iPS (FIG. 25).

20-5: Examination of Pluripotency of Pluripotent Stem Cells Induced by Nicotinamide

In order to examine whether the pluripotent stem cell lines (Nam-iPS) induced from human fibroblasts by the addition of nicotinamide possess differentiation potential that is the feature of stem cells, the differentiation potential of embryoid bodies derived from each of the induced pluripotent stem cell lines was examined. Specifically, the cells were cultured in suspension, and then the embryoid bodies were incubated again on gelatin-coated plates for 10 days under differentiation conditions, after which the expression of marker proteins that are expressed specifically in cells that differentiated into three germ layers was analyzed by RT-PCR and immunochemical staining. The expression pattern specific for each of three germ layers was analyzed by PCR using the primers shown in Table 1 above. As a result, it was shown that genes specific for exoderm (NCAM, PAX6, VIM, and OTX1), mesoderm (cTnT, IGF2, TBX6, and RUNX2) and endoderm (AMYLASE, HAND1, PECAM, and HGF) were all expressed (FIG. 26). In addition, as can be seen in the results of immunocytochemical staining in FIG. 27, cells positive to Tuj1 (exoderm), Nestin (exoderm), desmin (mesoderm), α-SMA (α-smooth muscle actin, mesoderm), Sox17 (endoderm) and FoxA2 (endoderm) were detected (FIG. 27). Such results that the pluripotent stem cell lines (Nam-iPS) induced by the addition of nicotinamide had the capability to differentiate into three germ layers and maintained pluripotency.

In addition, in order to examine the in vivo totipotency of the human pluripotent stem cell lines (Nam-iPS) induced by the addition of nicotinamide, the Nam-iPS-induced pluripotent stem cell lines were injected subcutaneously into the dorsal flanks of immunodeficiency (SCID) mice. After about 12 weeks, teratomas could be observed, and neural rosette (exoderm), meuronal epithelium (exoderm), adipocyte (mesoderm), smooth muscle (mesoderm), bone (mesoderm), cartilage (mesoderm), myxoid tissue (mesoderm) and gut-like epithelium (endoderm) were observed in the teratomas by hematoxylin/eosin staining (FIG. 28). This suggests that the human pluripotent stem cell lines (Nam-iPS) induced by the addition of nicotinamide have the capability to differentiate into three germ layers in vitro and in vivo.

### Example 21

### Measurement Low-Density Cloning Effect

In order to examine the effect of nicotinamide on the survival of human pluripotent stem cells at low density, human pluripotent stem cells were treated with TrypLE reagent (Invitrogen) for 3 minutes, and then dissociated into single cells by pipetting and seeded on a Matrigel-coated 96-well plate at a density of 500 cells per well. After 5 days of culture, human pluripotent stem cell colonies started to be formed, and the cells were fixed with 4% paraformaldehyde, and then AP staining was performed or the level of apoptosis was measured.

### Example 22

### Effect of Nicotinamide on Maintenance of Undifferentiated State of Pluripotent Stem Cells

In order to verify the effect of nicotinamide on the maintenance of undifferentiated state of pluripotent stem cells, the present inventors performed the following experiment. While human embryonic stem cells and human pluripotent stem cells were cultured in MEF-CM or UM on feeder-free Matrigel, various NAD precursors including nicotinamide were added to the stem cells, and then the effects of the NAD precursors on the stem cells were verified by examining the morphological change and the expression pattern of AP that is a human embryonic stem cell-specific marker. The human embryonic stem cells cultured in UM for 6 days did not maintain the morphology of undifferentiated cells, and the expression of AP in the cells was down-regulated, suggesting that the cultured human embryonic stem cells were in the initial stage of differentiation (FIG. 29). Particularly, in comparison with the case in which other NAD precursors were added, the human embryonic stem cells cultured in UM containing nicotinamide (1 mM) showed the morphology of undifferentiated human embryonic stem cells, and particularly, the size of colonies of the human embryonic stem cells was increased, the number of AP-positive (AP⁺⁾ cells was generally increased (FIG. 29).

In addition, in order to examine the effects of nicotinamide at various concentrations, various concentrations of nicotinamide were added to UM, and the degrees of maintenance of undifferentiation of human embryonic stem cells and human induced pluripotent stem cells, which are pluripotent stem cells, were examined. It was shown that the human embryonic stem cells and human induced pluripotent stem cells cultured in UM containing 0.1-1 mM of nicotinamide conserved the morphology of typical undifferentiated human embryonic stem cells and showed the positive expression of the human embryonic stem cell-specific marker AP (FIG. 29). Particularly, when 0.1 mM of Nam was added, the undifferentiated state was most effectively maintained, and the undifferentiated human embryonic stem cells were maintained in a state almost similar to that of the human embryonic stem cells and human induced pluripotent stem cells cultured in CM. However, it was shown that the addition of a low concentration (<0.01 mM) or high concentration (>5 mM) of nicotinamide could not maintain the undifferentiated state of pluripotent stem cells (FIG. 30).

The effect of nicotinamide effective for maintaining the undifferentiated state of human embryonic stem cells and human induced pluripotent stem cells, which are pluripotent stem cells, was verified under various conditions. First, when feeder cells were used, the number of AP-positive cells was most increased at a nicotinamide concentration of 0.1 mM (FIG. 31). In order to eliminate the use of feeder cell-derived factors and animal serum and ensure culture conditions including a medium (CDM) whose components are known, various concentrations of nicotinamide were added to mTeSR1 (CDM) without feeder cells, and the effects thereof were examined. As a result, it could be seen that the addition of 0.1 mM of nicotinamide was effective for maintaining the undifferentiated state of various human embryonic stem cell lines (H1 and H9) and the human induced pluripotent stem cell line (hiPS) (FIG. 31). Such results suggest that nicotinamide is effective as a medium additive for culturing and maintaining various pluripotent stem cells, including human embryonic stem cells and human induced pluripotent stem cells.

### Example 23

### Examination of Effects of Nicotinamide on Promotion of Proliferation of Pluripotent Stem Cells and on Improvement in Mitochondrial Function

In order to verify the effect of nicotinamide on the proliferation of pluripotent stem cells, a BrdU incorporation assay was performed. Nicotinamide was added to mTeSR1 (CDM) at various concentrations, and the effects thereof were examined. BrdU incorporation was significantly increased when nicotinamide was added at concentrations of 0.1 mM (1.6 times; FIG. 32) and 1 mM (1.2 times; FIG. 32), compared to that in the human embryonic stem cells cultured in mTeSR1.

In addition, the present inventors measured the membrane potential (ΔΨm) of mitochondria. When human embryonic stem cells were maintained and cultured, various concentrations of nicotinamide were added to mTeSR1, and then the mitochondrial membrane potential of the human embryonic stem cells was measured by JC-1 staining. After JC-1 staining, activated mitochondria are stained with red, and non-activated mitochondria are stained with green. The ratio of the number of red-stained mitochondria to the number of green-stained mitochondria is measured, and an increase in the ratio indicates an increase in the number of activated mitochondria. It was shown that the addition of 0.1 mM nicotinamide significantly increased the number of activated mitochondria (FIG. 33).

### Example 24

### Examination of the Increase in Reprogramming Efficiency Caused by Nicotinamide

In order to examine the change in reprogramming efficiency of human fibroblasts caused by nicotinamide, retrovirus expressing the reprogramming factors Oct4, Sox2, Klf4 and c-Myc was transfected at an MOI of 1. Reprogramming efficiencies caused by the addition of nicotinamide and other NAD precursors were examined. As a result, from the increased number of AP-positive colonies, it could be seen that the addition of nicotinamide increased the efficiency of reprogramming by about 17 times compared to the addition of other NAD precursors (FIG. 4A). The efficiency of reprogramming by nicotinamide increased in a manner dependent on the concentration of nicotinamide. From the increased number of AP-positive (AP⁺⁾ colonies, it could be seen that the efficiency of reprogramming increased by 13 times (0.1 mM), 28 times (1 mM), 16 times (10 mM) and 2 times (20 mM) compared to that of the untreated group (FIG. 4B).

The change in reprogramming efficiency according to the timing and period of treatment of nicotinamide was examined, and as a result, it was verified that treatment with nicotinamide in an initial stage immediately after viral infection most effectively increased the efficiency of reprogramming, and then additionally increased the efficiency of reprogramming in a time-dependent manner (FIG. 5).

### Example 25

### Effect of Nicotinamide on Acceleration of Reprogramming Kinetics of hiPSCs

It was found that nicotinamide increases the reprogramming efficiency of hiPSCs and also accelerates the reprogramming kinetics of hiPSCs. From day 5 of culture (D5), the expression levels of the stem cell markers Nanog and Tra-1-60 in the cell group treated with nicotinamide increased by 3 times and 8 times, respectively, compared to those in the untreated group (FIG. 6). In addition, the mRNA expression levels of the stem cell marker Nanog and the proliferation regulator TERT rapidly increased from day 7 by treatment with nicotinamide (FIG. 7). A period of time of 3 weeks or more is generally required until hiPSCs colonies having distinct boundaries while the cells are clustered, whereas the period was significantly reduced to 2 weeks or less when the cells were treated with nicotinamide (FIG. 8).

### Example 26

### Examination of Nicotinamide on Cell Proliferation

The inhibition of cell proliferation by introduction of the reprogramming factor OSKM is already known. The present inventors have found that nicotinamide promotes the proliferation of cells regardless of introduction of reprogramming factors. On days 19 and 26, it was observed that treatment with nicotinamide at concentrations of 0.1, 1 and 10 mM increased the number of healthy viable cells in a concentration-dependent manner (FIG. 10). Particularly, on day 19 of induction of reprogramming, it was observed that treatment with 0.1-1 mM of nicotinamide significantly increased the number of actually dividing cells incorporated with BrdU (FIG. 11). In addition, on day 12 of induction of reprogramming, it was shown by live cell imaging that treatment with 1 mM of nicotinamide increased the ratio of proliferating cells (FIG. 12).

### Example 27

### Examination of Effect of Nicotinamide on Inhibition of Cellular Senescence

The ratio of cells stained with senescence-associated beta-galactosidase increased to 18.1% on day 19 and 35.2% on day 26 by introduction of the reprogramming factor OSKM, and when cells were treated with 1 mM of nicotinamide, the ratio of the stained cells decreased by 88.1±1.4% and 76.9±2.5% on days 19 and 26, respectively, compared to that of the untreated group (FIG. 13A). Also, when cells were treated with 10 mM of nicotinamide, the number of senescent cells decreased by 76.1±1.4% on day 19 and 30.8±9.0% on day 21 compared to that of the control group (FIG. 13A). In addition, when cells were treated with 1 mM of nicotinamide, cellular senescence-associated DNA damage was also decreased 83.0±0.5% compared to that of the control group (FIG. 13B).

### Example 28

### Examination of Effect of Nicotinamide on Inhibition of Oxidative Stress

Cellular senescence by oxidative stress was previously reported. The present inventors observed that the formation of reactive oxygen species on day 19 of induction of reprogramming was increased by introduction of the reprogramming factor OSKM (FIG. 14) and that protein damage caused by oxidative stress was increased (FIG. 15). Moreover, it was shown that, when cells were treated with 1 mM of nicotinamide, the formation of reactive oxygen species was inhibited and the amount of damaged protein also decreased. In addition, it was observed that, in reprogramming conditions in which the formation of reactive oxygen species increases and protein is damaged by oxidative stress, the membrane potential of mitochondria was not maintained, whereas, when cells were treated with 1 mM of nicotinamide, the membrane potential was maintained (FIG. 16).

### Example 29

### Examination of Effect of Nicotinamide on Inhibition of Signaling of Senescence Factor p53

By immunostaining (FIG. 17), Western blot analysis (FIGS. 18 and 20), real-time PCR (FIG. 19) and the like in the reprogramming process, it was shown that the mRNA and protein expression levels of the senescence-associated signaling factors p53, p21 and p16 were significantly decreased by treatment with 1 mM of nicotinamide.

### Example 30

### Examination of Effect of Nicotinamide as Medium Additive for Maintaining Undifferentiated State

H9 human pluripotent stem cells generally differentiate in UM medium, but when 0.1 mM of nicotinamide was added, the cells could be maintained in an undifferentiated state without differentiation (FIG. 34A). In addition, the results of AP staining and apoptosis assay indicated that the addition of nicotinamide significantly increased the survival and self-renewal abilities of H9 human embryonic stem cells and human induced pluripotent stem cells, which are human pluripotent stem cells, even when the cells were cultured at low density after single-cell dissociation (FIG. 34B). Furthermore, the results of TRA-1-60 staining indicated that, even in differentiation media such as UM, the addition of 0.1 mM nicotinamide enabled stem cells to be subcultured for a long period of time for 10 or more passages in an undifferentiated state (FIG. 35). Also, normal karyotypes could be observed even after the long-term subculture (FIG. 36). Such results indicate that nicotinamide can control cellular and molecular mechanisms to improve conditions for maintaining and culturing human pluripotent stem cells in an undifferentiated state.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A composition for promoting reprogramming of differentiated cells into pluripotent stem cells, the composition comprising nicotinamide.

2. The composition of claim 1, wherein the differentiated cells are somatic cells or progenitor cells.

3. The composition of claim 1, wherein the composition is a culture medium.

4. The composition of claim 3, wherein a concentration of nicotinamide in the composition is 0.01-20 mM.

5. The composition of claim 1, wherein the composition comprises one or more reprogramming factors.

6. The composition of claim 5, wherein the reprogramming factors are proteins selected from the group consisting of Oct4, Sox2, K1F4, c-Myc, Nanog, Lin-28 and Rex1, or nucleic acid molecules encoding the proteins.

7. A method of producing reprogrammed pluripotent stem cells from differentiated cells, the method comprising the steps of:
(a) transferring a reprogramming factor to the differentiated cells; and
(b) culturing the differentiated cells in a medium containing the composition of claim 1.

8. The method of claim 7, wherein the differentiated cells are of human origin.

9. The method of claim 7, wherein the reprogramming of the differentiated cells into the pluripotent stem cells corresponds to an increase in growth and proliferation of cells, inhibition of apoptosis, an increase in mitochondrial activity, inhibition of senescence, a decrease in oxidative stress, inhibition of p53 signaling, a reduction in reprogramming time or an increase in reprogramming efficiency in reprogrammed cells compared to that in the differentiated cells.

10. The method of claim 7, further comprising a step of separating embryonic stem cell-like colonies from a culture resulting from step (b).

11. The method of claim 7, wherein steps (a) and (b) are performed simultaneously, sequentially or in the reverse order.

12. A method of culturing reprogrammed pluripotent stem cells in an undifferentiated state, the method comprising culturing reprogrammed pluripotent stem cells, produced by the method of claim 7, in a medium containing nicotinamide.

13. A composition for maintaining or improving the mitochondrial function of pluripotent stem cells, the composition comprising nicotinamide.

14. The composition of claim 13, wherein the mitochondrial function is measurable by membrane potential activity.

15. A composition for maintaining pluripotent stem cells in an undifferentiated state, the composition comprising nicotinamide.

16. The composition of claim 15, wherein the composition is a culture medium.

17. The composition of claim 16, wherein a concentration of nicotinamide in the composition is between 0.01 mM and 20 mM.

18. A method of culturing pluripotent stem cells so as to be maintained in an undifferentiated state, the method comprising culturing the cells using the composition of claim 15.

19. The method of claim 18, wherein the pluripotent stem cells are maintained in an undifferentiated state in the presence or absence of serum or feeder cells.

20. A method for preparing a cell culture, the method comprising culturing pluripotent stem cells using the composition of claim 15 so as to be maintained in an undifferentiated state.

21. The method of claim 20, wherein the culture is a plurality of continuous subcultures.

22. The method of claim 20, wherein the pluripotent stem cells are embryonic stem cells or induced pluripotent stem cells.


SEQUENCE LISTING (64 sequences)

SEQ ID NO: 1 - DNA, 23 bp, Artificial Sequence
  total OCT4 primer F
gagaaggatgtggtccgagtgtg

SEQ ID NO: 2 - DNA, 22 bp, Artificial Sequence
  total OCT4 primer R
cagaggaaaggacactggtccc

SEQ ID NO: 3 - DNA, 20 bp, Artificial Sequence
  total SOX2 primer F
agaaccccaagatgcacaac

SEQ ID NO: 4 - DNA, 20 bp, Artificial Sequence
  total SOX2 primer R
atgtaggtctgcgagctggt

SEQ ID NO: 5 - DNA, 20 bp, Artificial Sequence
  total KLF4 primer F
accctgggtcttgaggaagt

SEQ ID NO: 6 - DNA, 20 bp, Artificial Sequence
  total KLF4 primer R
acgatcgtcttcccctcttt

SEQ ID NO: 7 - DNA, 20 bp, Artificial Sequence
  total cMYC primer F
cctaccctctcaacgacagc

SEQ ID NO: 8 - DNA, 20 bp, Artificial Sequence
  total cMYC primer R
ctctgaccttttgccaggag

SEQ ID NO: 9 - DNA, 24 bp, Artificial Sequence
  Endo OCT4 primer F
gacagggggaggggaggagctagg

SEQ ID NO: 10 - DNA, 26 bp, Artificial Sequence
  Endo OCT4 primer R
cttccctccaaccagttgccccaaac

SEQ ID NO: 11 - DNA, 26 bp, Artificial Sequence
  Endo SOX2 primer F
gggaaatgggaggggtgcaaaagagg

SEQ ID NO: 12 - DNA, 26 bp, Artificial Sequence
  Endo SOX2 primer R
ttgcgtgagtgtggatgggattggtg

SEQ ID NO: 13 - DNA, 22 bp, Artificial Sequence
  Endo KLF4 primer F
agcctaaatgatggtgcttggt

SEQ ID NO: 14 - DNA, 23 bp, Artificial Sequence
  Endo KLF4 primer R
ttgaaaactttggcttccttgtt

SEQ ID NO: 15 - DNA, 15 bp, Artificial Sequence
  Endo cMYC primer F
cgggcgggcactttg

SEQ ID NO: 16 - DNA, 19 bp, Artificial Sequence
  Endo cMYC primer R
ggagagtcgcgtccttgct

SEQ ID NO: 17 - DNA, 20 bp, Artificial Sequence
  hTERT primer F
cggaagagtgtctggagcaa

SEQ ID NO: 18 - DNA, 19 bp, Artificial Sequence
  hTERT primer R
ggatgaagcggagtctgga

SEQ ID NO: 19 - DNA, 23 bp, Artificial Sequence
  Trans OCT4 primer F
gagaaggatgtggtccgagtgtg

SEQ ID NO: 20 - DNA, 24 bp, Artificial Sequence
  Trans OCT4 primer R
ccctttttctggagactaaataaa

SEQ ID NO: 21 - DNA, 26 bp, Artificial Sequence
  Trans SOX2 primer F
ggcacccctggcatggctcttggctc

SEQ ID NO: 22 - DNA, 24 bp, Artificial Sequence
  Trans SOX2 primer R
ttatcgtcgaccactgtgctgctg

SEQ ID NO: 23 - DNA, 25 bp, Artificial Sequence
  Trans KLF4 primer F
acgatcgtggccccggaaaaggacc

SEQ ID NO: 24 - DNA, 24 bp, Artificial Sequence
  Trans KLF4 primer R
ttatcgtcgaccactgtgctgctg

SEQ ID NO: 25 - DNA, 27 bp, Artificial Sequence
  trans cMYC primer F
caacaaccgaaaatgcaccagccccag

SEQ ID NO: 26 - DNA, 24 bp, Artificial Sequence
  trans cMYC primer R
ttatcgtcgaccactgtgctgctg

SEQ ID NO: 27 - DNA, 20 bp, Artificial Sequence
  NANOG primer F
caaaggcaaacaacccactt

SEQ ID NO: 28 - DNA, 20 bp, Artificial Sequence
  NANOG primer R
attgttccaggtctggttgc

SEQ ID NO: 29 - DNA, 20 bp, Artificial Sequence
  NCAM primer F
aggagacagaaacgaagcca

SEQ ID NO: 30 - DNA, 20 bp, Artificial Sequence
  NCAM primer R
ggtgttggaaatgctctggt

SEQ ID NO: 31 - DNA, 20 bp, Artificial Sequence
  PAX6 primer F
gccagcaacacacctagtca

SEQ ID NO: 32 - DNA, 20 bp, Artificial Sequence
  PAX6 primer R
tgtgagggctgtgtctgttc

SEQ ID NO: 33 - DNA, 20 bp, Artificial Sequence
  VIM primer F
gggacctctacgaggaggag

SEQ ID NO: 34 - DNA, 20 bp, Artificial Sequence
  VIM primer R
cgcattgtcaacatcctgtc

SEQ ID NO: 35 - DNA, 26 bp, Artificial Sequence
  OTX1 primer F
taaccctacgccctcctcttcctact

SEQ ID NO: 36 - DNA, 26 bp, Artificial Sequence
  OTX1 primer R
aagcagtcggcagagttgaaggcaag

SEQ ID NO: 37 - DNA, 22 bp, Artificial Sequence
  cTnT primer F
ggcagcggaagaggatgctgaa

SEQ ID NO: 38 - DNA, 23 bp, Artificial Sequence
  cTnT primer R
gaggcaccaagttgggcatgaac

SEQ ID NO: 39 - DNA, 20 bp, Artificial Sequence
  IGF2 primer F
cagacccccaaattatcgtg

SEQ ID NO: 40 - DNA, 20 bp, Artificial Sequence
  IGF2 primer R
gccaagaaggtgagaagcac

SEQ ID NO: 41 - DNA, 20 bp, Artificial Sequence
  TBX6 primer F
tacattcaccccgactctcc

SEQ ID NO: 42 - DNA, 20 bp, Artificial Sequence
  TBX6 primer R
tgtatgcggggttggtactt

SEQ ID NO: 43 - DNA, 20 bp, Artificial Sequence
  RUNX2 primer F
cactcactaccacacctacc

SEQ ID NO: 44 - DNA, 20 bp, Artificial Sequence
  RUNX2 primer R
gtcgccaaacagattcatcc

SEQ ID NO: 45 - DNA, 20 bp, Artificial Sequence
  HGF primer F
gcatcaaatgtcagccctgg

SEQ ID NO: 46 - DNA, 20 bp, Artificial Sequence
  HGF primer R
caacgctgacatggaattcc

SEQ ID NO: 47 - DNA, 23 bp, Artificial Sequence
  AMYLASE primer F
gctgggctcagtattccccaaat

SEQ ID NO: 48 - DNA, 24 bp, Artificial Sequence
  AMYLASE primer R
gacgacaatctctgacctgagtag

SEQ ID NO: 49 - DNA, 20 bp, Artificial Sequence
  HAND1 primer F
tgcctgagaaagagaaccag

SEQ ID NO: 50 - DNA, 20 bp, Artificial Sequence
  HAND1 primer R
atggcaggatgaacaaacac

SEQ ID NO: 51 - DNA, 20 bp, Artificial Sequence
  PECAM primer F
ccacatacactccttccacc

SEQ ID NO: 52 - DNA, 21 bp, Artificial Sequence
  PECAM primer R
gactacccaaaactacaagcc

SEQ ID NO: 53 - DNA, 19 bp, Artificial Sequence
  GAPDH primer F
gaaggtgaaggtcggagtc

SEQ ID NO: 54 - DNA, 20 bp, Artificial Sequence
  GAPDH primer R
gaagatggtgatgggatttc

SEQ ID NO: 55 - DNA, 25 bp, Artificial Sequence
  OCT4-1 primer F
atttgttttttgggtagttaaaggt

SEQ ID NO: 56 - DNA, 27 bp, Artificial Sequence
  OCT4-1 primer R
ccaactatcttcatcttaataacatcc

SEQ ID NO: 57 - DNA, 27 bp, Artificial Sequence
  OCT4-2 primer F
ggatgttattaagatgaagatagttgg

SEQ ID NO: 58 - DNA, 25 bp, Artificial Sequence
  OCT4-2 primer R
cctaaactccccttcaaaatctatt

SEQ ID NO: 59 - DNA, 25 bp, Artificial Sequence
  NANOG primer F
tggttaggttggttttaaatttttg

SEQ ID NO: 60 - DNA, 26 bp, Artificial Sequence
  NANOG primer R
aacccacccttataaattctcaatta

SEQ ID NO: 61 - DNA, 20 bp, Artificial Sequence
  Oct4 promoter primer F
ttgccagccattatcattca

SEQ ID NO: 62 - DNA, 20 bp, Artificial Sequence
  Oct4 promoter primer R
tatagagctgctgcgggatt

SEQ ID NO: 63 - DNA, 20 bp, Artificial Sequence
  Nanog promoter primer F
gatttgtgggcctgaagaaa

SEQ ID NO: 64 - DNA, 20 bp, Artificial Sequence
  Nanog promoter primer R
ggaaaaaggggtttccagag